(19) United States
(12) Patent Application Publication
(10) Pub. No.: US 2018/0228942 A1
(43) Pub. Date: Aug. 16, 2018

(54) A DELIVERY DEVICE

(71) Applicant: PTT HOLDING APS, Lyngby (DK)

(72) Inventors: Peter Theilade THOMSEN, Kgs. Lyngby (DK); Martin ALM, Greve (DK)

(73) Assignee: PTT HOLDING APS, Lyngby (DK)

(21) Appl. No.: 15/751,517

(22) PCT Filed: Aug. 11, 2016
(86) PCT No.: PCT/DK2016/050270

§ 371 Date: Feb. 9, 2018

(30) Foreign Application Priority Data
Aug. 11, 2015 (DK) PA 2015 70520

(51) Int. Cl. A61L 29/04; A61M 25/00; A61M 25/10; A61B 5/01; A61B 5/145; A61F 6/14; A61D 7/00; A61B 5/00; A61L 29/14; A61L 29/16; A61L 29/00; A61K 9/00; A61K 9/48; C02F 1/68

(52) U.S. Cl. CPC A61L 29/049; A61N 5/062; A61M 25/1002; A61B 5/01; A61B 5/14539; A61F 6/142; A61D 7/00; A61B 5/6852; A61L 29/145; A61L 29/16; A61L 29/005; A61K 9/0036; A61K 9/4866; C02F 1/685; A61M 2205/0216; A61M 25/0017

(57) ABSTRACT

The invention relates to a delivery device suitable for delivering a chemical substance, e.g. a medical device such as a catheter, a microcapsule, an implantable capsule or a P-ring, or a delivery device for use in the construction industries e.g. in the form of microcapsules comprising antifouling agent for marine paint. The delivery device comprises a closed cavity, the cavity is defined by an innermost wall surface, wherein at least a section of the inner wall surface constitutes an inner surface of a delivery membrane wherein the delivery membrane comprises an interpenetrating polymer network substrate comprising a host polymer and a guest polymer, where the guest polymer is interpenetrating the host polymer to form substantially continuous pathways within said host polymer.

### TECHNICAL FIELD

The invention relates to a delivery device suitable for delivering a chemical substance, e.g. a medical device such as a catheter, a microcapsule, an implantable capsule or a P-ring, or a delivery device for use in the construction industries e.g. in the form of microcapsules comprising antifouling agent for marine paint.

### BACKGROUND ART

A plurality of methods for delivering chemical substances such as chemical compounds or compositions is known in the art. In recent years large effort has been applied in developing delivery devices in the form of a polymer matrix, in particular hydrogels, wherein the chemical compound for release embedded in the matrix.

A study by Garcia et al. “5-Fluorouracil trapping in poly(2-hydroxyethyl methacrylate-co-acrylamide) hydrogels; in vitro drug delivery studies”, European Polymer Journal 36 (2000) 111-122, describes such delivery devices based on hydrogels where the 5-Fluorouracil was trapped in the gel during its polymerization.

In Ferreira et al. “Evaluation of poly(2-hydroxyethyl methacrylate) gels as drug delivery systems at different pH values”, International Journal of Pharmaceuticals 194 (2000) 169-180, the influence of the pH value during loading of Salicylic acid in PHEMA gel was tested. The loading was performed by immersing in a liquid comprising the salicylic acid at various pH values.

Karlgard et al. “In vitro uptake and release studies of ocular pharmaceutical agents by silicon-containing and p-HEMA hydrogel contact lens materials”, International Journal of Pharmaceuticals 257 (2003) 141-151 discloses a study of the uptake and release of different chemical compounds in different types of contact lenses. The loading was performed by soaking in a liquid containing the chemical compound. It was found that both the uptake and the release were relatively rapid.

Santos et al. “Poly(hydroxyethyl methacrylate-co-methacrylated-βcyclodextrin) hydrogels: Synthesis, cytocompatability, mechanical properties and drug loading/release properties” ScienceDirect, Acta Biomaterialia 4 (2008) 745-755, describes loading/release properties of hydrocortisone and acetazolamide in the hydrogel. The loading was performed by immersing in liquid containing the drug. It was found that a slow drug release could be obtained.

WO 2005/055972 discloses a drug delivery device comprising a polymeric matrix and a drug distributed within the matrix. The device is essentially free of solvents in particular organic solvents for the drug. The matrix may be an interpenetrating polymer network (IPN) of a large number of different polymer materials.

The drug delivery device is produced by loading the matrix with a drug using a drug carrier in the form of a gas, a supercritical fluid, a high pressure liquid, or a dense gas-like fluid. A preferred drug carrier includes CO₂ in a liquid and/or supercritical state. No specific methods are described.

CN103623497A describes a drug delivery balloon dilating catheter of a double-layer balloon structure. The drug delivery balloon dilating catheter comprises a pressurizing hole-free inner balloon body and an outer balloon body with a drug-permeable micro-hole permeable membrane. A similar balloon catheter is described in U.S. Pat. No. 5,569,184 where the inflating balloon is surrounded by a delivery balloon with delivery ports or of inherently permeable fabrics such that drug introduced into the interspace between the inflating balloon and the delivery balloon is immediately delivered via physical holes provided by the fabrics or the delivery ports.

CN101733051 describes a method of preparing PDMS/PHEMA IPN hollow microcapsules by a method where a mixture of PDMS, TEOS, stannous octoate and toluene is dispersed into PVA solution and cured to form microspheres of a first PDMS network. The first PDMS network spheres are hereafter soaked in a mixture of toluene, crosslinking agent and HEMA for some hours and crosslinked. It is considered that the spheres may be used for drug delivery.

US2014309598A describes an osmotically active intravaginal delivery system for the controlled release of therapeutically active substances into the vaginal cavity. The osmotically active vaginal delivery system has a body which comprises at least one compartment comprising a composition of one or more therapeutically active substances and at least one compartment comprising an osmotically composition capable of interacting with water and aqueous biological fluids to create a concentration gradient against the exterior fluid or to swell or expand to create osmotic pressure and thereby providing a pressure to release the active substances via a passageway extending from the compartment comprising the composition of one or more therapeutically active substances to the outer surface of the body.

US2012080378A describes forward osmosis membranes having a hydrophilic support layer and a polyamide rejection layer in a thin film composite membrane. Preferred support layer materials include aramid polymers and PVDF. A woven or non-woven mesh can be incorporated into the support layer to improve the handling properties of the membrane. Flat sheet and hollow fiber configurations are possible. Antifouling techniques are provided. The polyamide layer can be formed on the hydrophilic support layer by interfacial polymerization. Applications include forward osmosis and pressure retarded osmosis, such as industrial product and/or waste concentration, hydration bags, energy/pressure generation, and controlled delivery of chemicals (e.g. for pharmaceutical applications).

### DISCLOSURE OF INVENTION

It is an object of the invention to provide a new type of delivery device suitable for delivering a chemical substance which can be applied for controlled release over a relatively long period of time and which is both safe and can dose a chemical substance with high accuracy.

In an embodiment it is an object of the invention to provide a delivery device which can be applied for delivering chemical substances over a relatively long period of time, in a flexible manner and/or with a desired release profile, such as a 0-order kinetic release profile.

In an embodiment it is an object of the invention to provide a delivery device which can be applied for delivering chemical substances without the delivery device comprises undesired organic solvents, which is practically all organic solvents with ethanol as an exception.

These and other objects have been solved by the invention as defined in the claims and as described herein below.

It has been found that the invention and embodiments thereof have a number of additional advantages which will be clear to the skilled person from the following description.

The delivery device of the invention has been found to be suitable for many types of applications where delivery of chemical substances is desired. The delivery device provides a new concept for delivery of drugs and other chemical substances where the delivery device can be designed to have desired release profile or profiles for example constant release, persistent release, zero order release and/or sustained release.

The delivery device comprises a closed cavity. The cavity is defined by an innermost wall surface, wherein at least a section of the inner wall surface constitutes an inner surface of a delivery membrane. The delivery membrane comprises an interpenetrating polymer network (IPN) substrate comprising a host polymer and a guest polymer, where the guest polymer is interpenetrating the host polymer to form substantially continuous pathways within the host polymer.

The term “closed cavity” means herein that the delivery device is configured for holding an amount of liquid such as water within the cavity without leaking the liquid. The delivery device may comprise a closable opening or may be adapted for penetration for filling the chemical substance into the cavity. This will be described in further detail below.

The section of the inner wall surface that constitutes the inner surface of the delivery membrane may be any portion of the inner wall surface, such as the entire inner wall surface or merely a smaller part of the inner wall surface, such as about 1% or more, such as 10% or more, such as 25% or more, such as 50% or more of the inner wall surface. Interpenetrating polymer networks are well known in the art and further information about interpenetrating polymer networks and how such networks can be provided is for example described in US2015038613, WO 2005/055972 and/or WO 2013/075724.

Any term used in singular form should also be interpreted to include the plural form of the term, unless otherwise specified.

The term “substantially” should herein be taken to mean that ordinary product variances and tolerances are comprised.

In an embodiment, the delivery membrane has an outer surface opposite to the inner surface of the delivery membrane. The distance between the outer surface and inner surface defines the thickness of the delivery membrane. Advantageously, the substantially continuous pathways of the guest polymer within the host polymer extend from the inner surface to the outer surface of the delivery membrane. Thereby the chemical substance can migrate through the delivery membrane via the interpenetrating pathways. The skilled person can design the release profile of the delivery membrane by adjusting the amount of guest polymer in the host polymer and by designing the formation of the pathways. By preparing the host polymer with a surplus of residuals that can be extracted e.g. by liquid, dense or supercritical CO₂, the skilled person can obtain a desired interpenetrating polymer network structure of the host polymer. In an embodiment the delivery membrane may on its inner and/or outer surface comprise a coating of a material substantially identical to the material of the guest polymer. Thereby the release of the chemical substance can be increased.

In an embodiment the host polymer is prepared with a surplus of residuals that can be extracted by an organic solvent that swells the host polymer without dissolving it. Afterwards undesired remaining's of organic solvent may be extracted e.g. using ethanol which is usually an acceptable solvent.

The delivery membrane can in principle have any thickness, depending on the desired release profile. In principle the chemical substance will migrate through the guest polymer pathways of the delivery membrane by the shortest route, but by having a delivery membrane with a varying thickness, the release profile may be shaped for example such that when the chemical substance in the cavity is reduced due to migration into and through the membrane for release of the substance at the outer surface of the membrane, the membrane will still maintain a certain amount of chemical substance and thereby the release profile can be relatively constant over an increased period of time in particular where the membrane is relatively thick. Thus, the thicker the membrane the longer a substantially constant release profile may be obtained. In an embodiment the chemical substance also migrates through or via the surface of the host polymer. In another embodiment there is substantially no migration of the chemical substance through the host polymer. In an embodiment where the chemical substance comprises two or more different components, one of these components primarily migrates through the guest polymer and another of the components primarily migrates through the host polymer.

Depending on the substances to be released, the substances usually also have certain solvability in the guest and/or the host matrix and intra molecular forces between the guest polymer/the host polymer, and a selected substance to be released can also be applied to shape the release profile. In an embodiment the host polymer and/or guest polymer is selected in relation to the substance(s) to be released a to have a work of adhesion (Whc) at 25° C. of at least about 0 J/m2 where the loading fluid is water preferably as described in US2014303263.

Advantageously at least a section of the delivery membrane has a thickness from 1 μm to 1 cm, preferably from 10 μm to 1 mm. A too thin delivery membrane may have too little mechanical toughness whereas the thicker the membrane the longer it will take for the chemical substance to migrate through the delivery membrane. Advantageously the delivery membrane is resilient and may be stretched when the cavity is filled with the chemical compound(s), and in this embodiment the thickness in non-stretched state may be selected to be I the larger end of the before mentioned ranges since the thickness will decrease as the delivery membrane is stretched.

The delivery membrane can be uniform or non-uniform. In an embodiment the delivery membrane has a substantially uniform thickness. In general a uniform thickness of the delivery membrane is simpler to design and produce.

The host polymer can in principle be any kind of polymer which is not dissolved by the chemical substance e.g. aqueous chemical substance and which has a sufficient mechanical toughness for the intended use of the delivery device. In general the purpose of the host polymer is to ensure sufficient mechanical toughness of the delivery membrane.

Generally it is desired that the strength of the membrane is mainly or fully provided by the host polymer. Thus in an embodiment the host polymer has a tear strength substantially identical to the membrane.

In an embodiment the host polymer is a cross-linked polymer, such as a cross-linked elastomer e.g. thermoplastic elastomer (TPE), polyolefin elastomer (POE), polyurethane (PU), rubber e.g. latex rubber, silicone or any combinations thereof.

In an embodiment the host polymer is physically cross-linked, the host polymer is preferably a physically cross-linked TPE. The physically cross-linked TPE comprises physically cross-linked stabilizing domains that are reversible, and can be reformed e.g. by heat or ion-exchange. The stabilizing domains may be non-crystalline or crystalline.

In an embodiment the host polymer is chemically cross-linked, the host polymer is preferably covalently cross-linked.

In an embodiment the host polymer is cross-linked by ionic bonds.

In an embodiment the rubber is natural rubber or synthetic rubber, such as a vulcanized polymer of isoprene, the rubber is preferably a silicone rubber or cross-linked Polyurethane.

In an embodiment the host polymer is an elastomer. Suitable elastomers include, thermoplastic elastomers (TPEs) polyolefin elastomers (POEs), thermoplastic polyurethane (TPU), rubber e.g. latex rubber, silicones and/or any combinations thereof. Elastomeric host polymers are particularly preferred in embodiments where the delivery membrane should have elastomeric properties e.g. where the delivery membrane is a balloon or a part of a balloon of a catheter.

In a preferred embodiment, the host polymer comprises thermoplastic polyurethane (TPU).

Suitable TPUs include for example the TPUs marketed by Lubrizol under the tradenames Carbothane™, Isoplast®, Pellethane®, Tecofle™, Tecophillic™ and Tecothanetm.

TPU is a linear segmented block copolymer composed of hard and soft segments.

The hard segment can be either aromatic or aliphatic. Aromatic TPUs are based on isocyanates such as MDI while aliphatic TPU's are based on isocyanates like H12 MDI. When these isocyanates are combined with short-chain diols, they become the hard block. Normally it is aromatic, but when colour and clarity retention in sunlight exposure is a priority, an aliphatic hard segment is often used.

TPU and compounds comprising TPU are very suitable. It is relatively simple to process and can contain large amount of guest polymer. Further TPU is highly elastic and has high resilience. The TPU comprises soft segments which advantageously can be of polyether or polyester type. The TPU is preferably polyether-based.

In an embodiment the host polymer comprises at least 10%, such as at least 20%, such as at least 40%, such as at least 60% by mass of the host polymer with a backbone consisting of Si and O atoms or consisting of Si atoms, the host polymer preferably comprises poly(dimethyl siloxane), poly(methylphenyl siloxane), fluorosilicone rubber, silicone esters, polysiloxanes, polysilanes, polychlorosilanes, polyalkoxysilanes, polyaminosilanes, polysilanes, polydialkylsiloxanes, polysiloxanes containing at least one phenyl substituent, vinyl-functionalized silicone, partially or fully fluorinated silicone or a mixture of two or more of the mentioned silicones.

In a preferred embodiment the host polymer comprises silicone.

It has been found that the structure of the host polymer may have large influence on the release profile and in particular to obtain a desired release profile. In an embodiment the host polymer has a structure of a network of strand shaped filaments comprising a plurality of intrastrand pathways. Such host polymer structure of a network of strand shaped filaments comprising a plurality of intrastrand pathways may for example be obtained by preparing the host polymer with a surplus of residuals prior to crosslinking and after crosslinking removing at least a part of such residuals e.g. by supercritical fluid extraction or near supercritical fluid extraction e.g. using CO₂ as extraction fluid. In an embodiment the extraction of residuals from the host polymer is performed using dense CO₂ gas extraction.

In an embodiment the host polymer is prepared with a surplus of residuals that can be extracted by an organic solvent that swells the host polymer without dissolving it. Afterwards undesired remaining's of organic solvent may be extracted e.g. using ethanol which is usually an acceptable solvent. The organic solvent may be selected in depends of its ability to swell the host polymer and the time allowed for the swelling may also be used to regulate the removal of residuals from the host polymer.

In an embodiment the host polymer has a structure of a network of substantially flat strand shaped filaments. The phrase “substantially flat strand shaped filaments” means herein that the strands has a largest cross sectional dimension (perpendicular to a length dimension of the strand) which is much larger, such as at least 100% larger, such as from about 200% to 1000% larger than a shortest cross sectional dimension. The largest cross sectional dimension may be substantially perpendicular to the shortest cross sectional dimension.

In an embodiment the intrastrand pathways has a volume of at least about 30% of the total volume of the delivery membrane, such as at least about 40%, such as at least about 50%, such as at least about 60% of the total volume of the delivery membrane. The intrastrand pathways volume may for example be determined visually by freezing the host polymer and observing the structure in a microscope, such as a scanning electron microscope (SEM). In an embodiment the intrastrand pathways volume may for example be determined by weight by determine the weight of the host polymer prior to residual extraction and after residual extraction.

The volume of the intrastrand pathways may be used for designing the release profile. It is believed that he larger the intrastrand pathways volume the higher amount of drug may pass through the delivery membrane.

In an embodiment the host polymer and/or the guest polymer is electrically conductive.

In an embodiment the host polymer and the guest polymer have matching refractive index for at least one wavelength, such as for at least one wavelength in the range from 400 nm to 2 μm, such as in the visible range.

In an embodiment the host polymer has a higher refractive index than the guest polymer.

In an embodiment the host polymer and the guest polymer have substantially identical refractive index.

The host polymer may for example have a refractive index of from about 1.33 to about 1.5 and the guest polymer may for example have a refractive index of from about 1.1 to about 1.5. The guest polymer is selected to be suitable for the chemical substance to migrate in the guest polymer pathways. Where the chemical substance is alcohol-soluble, the guest polymer should advantageously have a high alcohol swelling property. The guest polymer may e.g. be an alcogel or a dried alcogel.

Advantageously, the chemical substance is soluble in aqueous media and the guest polymer has a high water swelling property.

In an embodiment the guest polymer comprises a gel selected from hydrogel, aerogel, xerogel or any combinations thereof.

Where a dry gel is used the dry gel will be re-moistured either by a user prior to using the delivery device, or it will be re-moistured during use by liquid from the liquid in which the chemical substance is dissolved and/or by mucosa liquid (bodily fluids).

In an embodiment the guest polymer comprises a homopolymer, preferably polymerized from an acrylate monomer or a vinyl polymer, more preferably n-vinyl pyrolidone (nVP), styrene; oxygen-, phenyl-, amino- and nitrogen-containing acrylic and methacrylic derivatives, e.g. acrylic esters, acrylic acids, methacrylic acid and -esters, alkyl and hydroxyalkyl acrylates and methacrylates; functionalized methacrylates such as 2-hydroxyethyl methacrylate (HEMA), glycerol monomethacrylate (GMMA), heptaflurobutyl acrylate (HFBA), 2-methacryloyloxyethyl phosphorylcholine (MPC) and [2-(methacryloyloxy)ethyl]-dimethyl-(3-sulfopropyl)-ammonium hydroxide (Betain); alkyl substituted acrylates and methacrylates such as methyl methacrylate (MMA), ethyl methacrylate (EMA), butyl methacrylate (BMA), dodecyl methacrylate (DMA); PEGylated (meth)actylates such as poly(ethylene glycol) methyl ether methacrylate (PEGMEMA) and poly(ethylene glycol) methyl ether acrylate (PEGMEA); substituted β- and γ-lactones, lactic acid monomers; carbohydrides and fluorinated monomers; urethanes; mono- and di-functional alcohols; carboxylic acids; amines; isocyanates; epoxides;

aromatics carrying alkyl group(s); sulfonated aromatics, aromatic resins; imidazole; imidazol derivatives; zwitterionic monomers; pyrazoles; quaternary ammonium monomers and any combinations thereof.

Examples of suitable homopolymers also include Nipam (N-Isopropylacrylamide) and pectin.

In an embodiment the guest polymer comprises a copolymer, preferably polymerised from monomers comprising silanes, e.g. tetraethylorthosilicate or tetraethoxysilane (TEOS), an acrylate monomer or a vinyl polymer, more preferably n-vinyl pyrolidone (nVP), styrene; oxygen-, phenyl, amino and nitrogen-containing acrylic and methacrylic derivatives, e.g. acrylic esters, acrylic acids, methacrylic acid and -esters, alkyl and hydroxyalkyl acrylates and methacrylates; functionalized methacrylates such as 2-hydroxyethyl methacrylate (HEMA), glycerol monomethacrylate (GMMA), heptaflurobutyl acrylate (HFBA), 2-methacryloyloxyethyl phosphorylcholine (MPC) and [2-(methacryloyloxy)ethyl]-dimethyl-(3-sulfopropyl)-ammonium hydroxide (Betain); alkyl substituted acrylates and methacrylates such as methyl methacrylate (MMA), ethyl methacrylate (EMA), butyl methacrylate (BMA), dodecyl methacrylate (DMA); PEGylated (meth)actylates such as poly(ethylene glycol) methyl ether methacrylate (PEGMEMA) and poly(ethylene glycol) methyl ether acrylate (PEGMEA); substituted β- and γ-lactones, lactic acid monomers; carbohydrides and fluorinated monomers; urethanes; mono- and di-functional alcohols; carboxylic acids; amines; isocyanates; epoxides; aromatics carrying alkyl group(s); sulfonated aromatics: aromatic resins; imidazole; imidazol derivatives; zwitterionic monomers; pyrazoles; quaternary ammonium monomers and any combinations thereof.

Copolymers mean polymers that are obtained by copolymerization of two or more monomer species.

In an embodiment the copolymer guest polymer is a bipolymer having two monomer species.

In an embodiment the copolymer guest polymer is a terpolymer obtained from three monomer species.

In an embodiment the copolymer guest polymer is a quaterpolymer obtained from four monomer species.

In an embodiment the guest polymer comprises poly(2-hydroxyethyl methacryate) (PHEMA), preferably the guest polymer comprises a copolymer of poly(2-hydroxyethyl methacryate) (PHEMA) and PEGMEA.

In an embodiment the guest polymer has a max. water swelling at 25° C. of about 10-10000% by mass of its dry mass, such as of about 10-5000% by mass of its dry mass, such as of about 10-500% by mass of its dry mass, such as of about 100-500% by mass of its dry mass.

In an embodiment the guest polymer has a max. water swelling at 25° C. of its dry mass which is higher than the max. water swelling at 25° C. of the host polymer.

In an embodiment the guest polymer has a max. water swelling at 25° C. of its dry mass which is equal to or lower than the max. water swelling at 25° C. of the host polymer.

In an embodiment the delivery membrane has a max. water swelling at 25° C. of about 5-5000% by mass of its dry mass, such as of about 10-1000% by mass of its dry mass, such as of about 10-500% by mass of its dry mass, such as of about 10-40% by mass of its dry mass.

Depending on the specific requirements which are desired to be met by the delivery device to be provided and the chemical substance to be applied and the desired release profile, the skilled person may find the optimal swelling degree and a suitable host and guest polymer by performing a number of tests, based on the above teaching.

It has been found to be very beneficial to provide the guest polymer to have a structure comprising a plurality of beads forming the substantially continuous pathways within the host polymer. By loading the guest monomer into the host polymer using a solvent in its supercritical, near supercritical or dense gas state the resulting guest polymer will form beads like units within the host polymer structure when it precipitates.

The precipitation either occurs during polymerization (if the polymer is not soluble in the polymerization solution) or when the solvent is removed. Advantageously, the supercritical, near super critical or dense gas solvent used comprises CO₂ or a combination of CO₂ with ethanol, such as up to 5% by mol. of ethanol. It is believed that the bead shape is provided when the polymerized monomers reaches a certain size and/or when the polymerized monomers is subjected to a pressure decrease when the pressure of the supercritical, near supercritical or dense gas is released and the supercritical, near supercritical or dense gas solvent used evaporates instantly and leaves the polymerized guest polymer within the holt polymer structure.

In an embodiment the plurality of beads are arranged in side-by-side formations to form the substantially continuous pathways within the host polymer. It has been found that where the amount of guest polymer is sufficient the beads of guest polymer may form side-by-side formations when the guest is loaded into the host polymer using a solvent in its supercritical, near supercritical or dense gas state.

In an embodiment the host polymer has a structure of a network of strand shaped filaments comprising a plurality of intrastrand pathways and the plurality of beads of guest polymers are arranged along the strand shaped filaments to form substantially continuous pathways within the host polymer. This intrastrand pathways structure may be as described above. It has been found that where silicone is used as host polymer and the residual oils is removed e.g. using a solvent in supercritical, near super critical or dense gas state, the guest polymer may be arranged along the strand shaped filaments and the plurality of beads of guest polymers may adhered to the strand shaped filaments to form the substantially continuous pathways within the host polymer.

In an embodiment the host polymer has a structure of a network of strand shaped filaments comprising a plurality of intrastrand pathways and the guest polymer is provided by soaking the host polymer in a solution of the selected monomers and optional polymerization initiator where the solvent used comprises an organic solvent that has the ability to swell the host polymer and thereby introduce the monomers and polymerization initiator into the network matrix of the host polymer provided by the network of strand shaped filaments. After a selected soaking time the solvent is allowed to evaporate and the polymerisation of the monomers is initiated e.g. by irradiation (e.g. using UV or IR) or by heating e.g. in an oven—e.g. to 70-90 ° C. After polymerization undesired remaining's of organic solvent may be extracted e.g. using ethanol which is usually an acceptable solvent. The polymerized guest polymer will thereafter be arranged as a surface coating onto the strand shaped filaments of the host polymer.

In an embodiment the delivery membrane comprises a continuous matrix of the host polymer and a plurality of interconnected paths of the guest polymer, wherein the interconnected paths of the guest polymer preferably extend through the whole thickness of the membrane to thereby provide suitable migration paths for the chemical substance through the delivery membrane.

In an embodiment the delivery membrane comprises a continuous matrix of the host polymer and a plurality of interconnected paths of the guest polymer, the delivery membrane has an outer surface and a plurality of paths of the guest polymer coincide with the inner surface and/or the outer surface. Thereby suitable migration paths for the chemical substance through the delivery membrane are provided.

In order to reduce delay (lag-phase) of release the delivery membrane may comprise one or more incorporated chemical compounds which may be equal to or differ from the chemical substance to be applied in the cavity.

In an embodiment the delivery membrane comprises one or more chemical compounds incorporated in the form of water-soluble particles, such as drugs, buffers, surfactants, fragrances, dyes, flavours, antioxidants, nutrients, hormones, catalysts and/or any combinations thereof. The chemical compound may advantageously be loaded into the delivery membrane by the method described in WO2013/075724.

Advantageously the delivery device is free of organic solvent other than ethanol. This may e.g. be obtained by using the method of loading the guest polymer into the host polymer as described in WO2013/075724.

In an embodiment the delivery membrane comprises one or more antimicrobial coatings. Such antimicrobial coatings normally prevent or reduce the growth of selected microorganism for a certain time, such as a few days, e.g. 3-5 days. In an embodiment where the delivery membrane has a lag-phase for the substances to migrate through the membrane via the paths of the guest polymer of 1 or more days, e.g. 2-5 days it is particularly desirable that the delivery membrane comprises one or more antimicrobial coatings on its outer side, opposite to its innermost wall surface.

To provide an effective loading of chemical substance into the cavity the delivery device should advantageously have a substantially size e.g. such that the chemical substance may be loaded into the cavity via a lumen formed in the delivery device and/or by using a needle to penetrate the membrane. Advantageously the membrane has a sufficiently elasticity to close such penetration hole after removing the needle.

Advantageously the inner surface of the delivery membrane has an inner surface area of at least about 0.01 cm², such as at least about 0.1 cm², such as at least about 1 cm², such as at least about 2 cm², such as at least about 5 cm², such as at least about 10 cm². Thereby it may be possibly to load the chemical substance using a needle as described above. Where the delivery device is entirely provided by the delivery membrane the delivery membrane needs to have a larger inner surface area, such as at least about 2 cm², such as at least about 5 cm², such as at least about 10 cm².

In an embodiment the cavity and the inner surface of said delivery membrane is sufficiently large to load drug into said cavity via a loading lumen and/or a needle.

Advantageously the cavity is sufficiently large to load at least 1 ml of fluid into said cavity.

In an embodiment the cavity is an at least partially collapsed cavity.

In an embodiment the wall comprising the innermost wall surface is inflatable, in other words, the delivery membrane is inflatable or stretchable, e.g. as an inflatable balloon or a section of an inflatable balloon of a balloon catheter.

In an embodiment the cavity comprises gas, preferably the wall comprising the innermost wall surface comprises a gas escape valve, such as a pressure relief valve to thereby adjust the amount of gas, e.g. when a chemical substance is loaded into the cavity.

Advantageously the cavity is adapted for being filled and/or refilled with one or more chemical substance in the form of fluids and or particles, such as cells, catalysts, drugs, buffers, surfactants, fragrances, dyes, flavours or any combinations thereof, the cavity is preferably adapted for being filled with one or more chemical substance by injecting via an inlet comprising a valve, such as an inflation lumen (e.g. of a Foley catheter) and/or by injection directly through the delivery membrane.

In an embodiment where the delivery membrane comprises an elastomer host polymer, the chemical substance can advantageously be injected into the cavity via the delivery membrane, and after removing the needle, the elastic properties of the delivery membrane ensure that the penetration hole made by the injection needle immediately contracts and fully closes/heals.

The chemical substance is advantageously in the form of one or more chemical compounds, preferably soluble in water or an aqueous solution, e.g. comprising buffer and other suitable additives.

In an embodiment the cavity comprises chemical substance(s) in the form of one or more chemical compounds in the form of fluids and or particles, such as cells, catalysts, drugs, buffers, surfactants, fragrances, dyes, flavours or any combination thereof.

In an embodiment the chemical substance comprises a drug, such as an active pharmaceutical ingredient (API). Preferably the drug is selected from antifungal, anticonvulsants, analgesics, antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids sympathomimetics, parasympathomimetics, tranquilizers, urinary tract drugs, vaccines, vaginal drugs, vitamins, collagen, hyaluronic acid, nonsteroidal anti-inflammatory drugs, angiotensin converting enzymes, polynucleotides, polypeptides, polysaccharides, nicotine, adreanal hormones, painkillers, morphine, anticancer and combinations and mixtures thereof.

In an embodiment the chemical substance comprises drug for treating symptoms of alzheimers, such as T3D-959, donepezil, galantamine, memantine, rivastigmine or combinations thereof. T3D-959 is a dual agonist of the peroxisome proliferator activated nuclear receptor delta/gamma, aka PPARδ/γ.

In an embodiment the chemical substance comprises a drug selected from components of the blood clotting cascade, such as factor VIII; birth control drugs, such as levonorgestrel or ethinyl estradiol; anti-virus drugs (antiretroviral), such as anti-HIV drugs (dapivirine, tenofovir) or anti-hepatitis C (ribavirin).

In an embodiment the chemical substance comprises a compound for human nutrition, such as a vitamin, a protein and/or a mineral or a component comprising one or more of the above-mentioned.

In an embodiment the chemical substance comprises a compound for microbiological nutrition, such as FeSO₄.7H₂O, ZnSO₄, KCI, MgSO₄, 7H₂O, NaNO₃, Glucose, NaCI, K₂HPO₄, NH₄H₂PO₄, CaCl₂.2H₂O, FeCl₃.6H₂O, NaH₂PO₄, Na₂HPO₄, NaHCO₃, (NH₄)₂SO₄ or mixtures comprising any of the mentioned.

In an embodiment the chemical substance comprises a fragrance compound, a flavor compound and/or a colour compound, e.g. pigment and/or dyes. By applying a colour compound in the chemical substance, the front of the migration of the chemical substance as it migrates through the delivery membrane may be visible.

In an embodiment the chemical substance comprises an active chemical compound and a colour compound, where the colour compound is selected such that its colour depends on the concentration of the active chemical compound, thereby the amount of remaining active chemical compound can be observed externally or internally to the membrane.

In an embodiment the chemical substance comprises a colour compound, where the colour compound is selected such that its can be applied as an indicator for a diagnosis.

Examples of further suitable chemical compound drugs include Anti-cancer drugs, such as Anastrozol, Bendamustine hcl, Bleomycinsulfate, Capecitabine, Cisplatin, Doxirubicinhcl Docetaxeltrihydrate or Gemcitabine hcl, Letrozole; Steroid Hormone drug, such as Dexamethasone, Estradiol, Fluocinolone acetonide, Gestodene, Hydrocortisone, Mifepristone, Mometasone furoate, Progesterone or Prednisolone, Triamcinolone Acetonide; Nootropic and Nutrition drug, such as Aniracetam, Acetyl L-Carnitine, Ademetionine, Disulfate, Tosylate (SAMe), Ademetionine1,4butanedisulfonate, Branched-chain amino acids, (BCAA), CDP Choline, Creatine Monohydrate, DHA EPA EGCG, L-Theanine, 1.3-dimethylamylamine, hcl (DMAA), Oxiracetam, Phenibut, Pramiracetam or Sulbutimiane; Cardiovascular system drug, such as Amlodipine besylate, Alagebrium chloride, Atorvastatin Calcium, Candesartan, Candesartan Cilexetil, Losartan potassium, Phentolamine mesylate, Rosuvastatin calcium or Telmisartan; Anti-depressant drug, such as Tianeptine acid, Tianeptine Sodium or Sertraline HCI; Antibiotics and anti-inflammatory drug, such as Amikacin sulphate, Chlorhexidine Collistin, Cyclosporin A, Daptomycin, Gatifloxacin, Gentamycin sulphate, Loxoprofen Sodium, Marbofloxacin, Nitrofurazone, Nitrofurantoin Polymyxin B sulfate, Rapamycin, Rifampicin Rifaximin, Sulphamethizole,Tacrolimus, Trichlosan or Tobramycin (Sulfate); Anti-fungal drug, such as miconazole; Prostaglandin drug, such as Alprostadil (PGE1), Bimatoprost, Dinoprost, trometamol (F2alpha), DL-Cloprostenol sodium, Latanoprost or Misoprostol; Cosmetic peptide drug, such as Argireline Acetate, Acetyl Octapeptide-3, Acetyl, Tetrapeptide-5, Copper peptide/Cu-GHK/AHK, Dipeptide-2/Pal-tetrapeptide-3, Hexapeptide-9, Hexapeptide-10, L-Carnosine, Matrixyl Acetate, Myristoyl, Pentapeptide-16, Myristoyl Pentapeptide-17, N-Acetyl carnosine, Octapeptide 2(TM-8-NH2), Palmitoyl tripeptide-1(Pal-GHK) or Palmitoyl Tripeptide-5; Urinary System drug, such as Finasteride, Dutasteride, Hydrochlorothiazide, Mirabegron, Oxbutynin, Spironolactone or Tamsulosin; Other drugs, such as Aprepitant, Benserazide hcl, Citicoline Sodium, Daclatasvir, Dextromethorphan HBr, Donepezil HCI, Entecavir, Fingolimod HcI, Flavoxate HCL, Formoterol Fumarate, Indocyanine Green, Ipratropium Bromide, Ketanserin tartrate, Ledipavir, Lorcaserin hydrochloride, Lactobionic acid, Levothyroxine sodium, Monoxidil, Obeticholic acid, Phenolphthalein, Pioglitazone HCI, Pimobendan Pirfenidone, Pregabalin, Sofosbuvir, Tadalafil, Tamoxifen citrate, Tenofovir, disoproxil fumarate, Tiotropium bromide, Tranexamic acid, Ulipristal acetate or Xylometazoline hcl.

In order to have a relatively high release it is desired that the molar mass of the chemical compound of the chemical substance is not too high. In an embodiment the chemical compound has a molar mass of up to about 300,000 g/mol., such as up to about 90.000 g/mol., such as up to about 50.000 g/mol., such as up to about 10.000 g/mol., such as up to about 5000 g/mol.

In an embodiment the chemical compound comprises particles selected from stem cell(s), catalyst(s), nanoparticle(s) or combinations thereof.

In an embodiment the cavity comprises particles comprising cells, such as stem cells or microorganisms. The cells are encapsulated in the cavity comprising the delivery membrane and are preferably capable of producing at least one by-product. The by-product is capable of migrating through the delivery membrane, preferably via pathways of the guest polymer when the delivery membrane is in wet condition, i.e. when the guest polymer is in moisture condition.

In an embodiment the delivery membrane comprises one or more chemical compounds in the form of fluids and or particles, such as cells, catalysts, drugs, buffers, surfactants, fragrances, dyes, flavours or any combinations thereof.

Advantageously the guest polymer is an aerogel or a xerogel of a hydrogel, preferably obtained by freeze drying the hydrogel or by freeze drying the delivery membrane with hydrogel guest polymer under supercritical or sub-supercritical conditions using a carrier gas such as CO₂.

Advantageously the chemical substance is selected relative to the membrane such that it diffuses through the membrane at zero order kinetics.

In an embodiment the chemical substance comprises drug trapped in liposomes which are dispersed in a polar liquid, preferably in an aqueous liquid. The liposomes comprise the drug to be released in a solution or dispersion trapped within the respective liposomes. Upon heating the lipid bilayer of the liposome will be ruptured and the drug will be released from the liposome. After being released from the liposome the drug will pass through the delivery membrane to be released from the delivery device with a desired release profile.

The liposome size can vary from very small (0.025 μm) to large (2.5 μm) vesicles.

Moreover, liposomes may have one or bilayer membranes. On the basis of their size and number of bilayers, liposomes can be classified into one of two categories: (1) multilamellar vesicles (MLV) and (2) unilamellar vesicles. Unilamellar vesicles can also be classified into two categories: (1) large unilamellar vesicles (LUV) and (2) small unilamellar vesicles (SUV) [16]. In unilamellar liposomes, the vesicle has a single phospholipid bilayer sphere enclosing a drug in solution or dispersion. In multilamellar liposomes, vesicles have an onion structure and several unilamellar vesicles may form on the inside of the other with smaller size, making a multilamellar structure of concentric phospholipid spheres separated by layers of drug in solution/dispersion.

In an embodiment the chemical substance comprises drug trapped in liposomes and the rupturing of the lipid bilayer of the liposome is provided by heating applied by irradiation, light or conventional heat.

In an embodiment the chemical substance comprises drug trapped in liposomes together with metal particles, preferably gold particles and the and the rupturing of the lipid bilayer of the liposome is provided by irradiating—e.g. by laser light or IR light, the metal particle thereby heating the particles and liposomes close to a heated metal particle will have its lipid bilayer ruptures such that the trapped drug is released from the liposome.

In an embodiment the chemical substance comprises drug trapped in liposomes together with metal particles, preferably gold particles, and together with light emitting units which light emitting units can be triggered to emit light to heat the metal particles. The rupturing of the lipid bilayer of the liposome is provided by triggering the light emitting unit thereby heating the particles and liposomes close to a heated metal particle will have its lipid bilayer ruptures such that the trapped drug is released from the liposome.

The light emitting units may e.g. be quantum dots often referred to as Q-dots. Examples of quantum dots are described in U.S. Pat. No. 7,498,177 and the quantum dots available from Life Technologies Europe BV. include more than 150 different product configurations with emission wavelength spanning in a broad wavelength range for examples quantum dots with the respective emission wavelengths: 525, 545, 565, 585, 605, 625, 655 and IR 705 and 800 nm. The quantum dots may be excited by irradiating from outside of the delivery device e.g. by laser light or IR light.

In an embodiment the light emitting units are light emitting diodes, such as the micro light-emitting diodes (μ-ILED array) described in “Wireless Optofluidic Systems for Programmable In Vivo Pharmacology and Optogenetic” by Jae-Woong Jeong et al. Cell 162, 662-674, Jul. 30, 2015 Elsevier Inc. DOI: http://dx.doi.org/10.1016/j.cell.2015.06.058

As described herein the micro light-emitting diodes may be triggered and even programmed wireless.

In an embodiment the chemical substance comprises drug trapped in liposomes together with micro light-emitting diodes as described above—i.e., without metal particles and the micro light-emitting diodes is triggered or even programmed by wireless connection.

In an embodiment the delivery device comprises a coating on at least a part of its inner or outer surface. In an embodiment also another part or parts of the delivery device comprise(s) a coating. In an embodiment the coating is selected from an adhesive, such as silicone adhesive, pressure sensitive adhesive, PU adhesive or TPU skin adhesive e.g. as marketed by MaterialScience. Baymedix. In an embodiment the coating is a biopolymer, such as Hyaluronic acid (HA), chitosan, lignin, alginate, pectin or combinations thereof. In an embodiment the coating is a hydrophilic coating e.g. a hydrogel coating e.g. for reducing friction against skin or mucosa. Preferably at least a part the inner surface and/or the outer surface of the delivery membrane comprises a coating.

In an embodiment the coating is a noble metal coating (i.e. bactiguard coating) or other drug delivering coatings, nitrofurazone, antibiotic silver, etc.

In an embodiment the coating is a hydrophobic coating (e.g. silicone oil)—e.g. for reducing friction from insertion.

In an embodiment the delivery device comprises a coating on at least a part of its inner or outer surface where the coating is made of a material substantially identical to the guest polymer.

In an embodiment the delivery device is a balloon catheter comprising at least one balloon forming the cavity and advantageously the delivery membrane is a part of or the entire balloon of the balloon catheter.

In an embodiment the delivery device is a balloon catheter as described in U.S. Pat. No. 6,602,243 with the difference that at least a section of the wall of the balloon is a delivery membrane as described herein.

Advantageously the delivery device is a urinary catheter, such as a Foley catheter and the delivery membrane is the balloon or a section thereof, the balloon is adapted to being inflated using an inflation liquid comprising the chemical compound(s) of the chemical substance. After the inflation liquid comprising the chemical compound(s) has been used as inflating liquid the chemical compound(s) will migrate through the delivery membrane.

Patients using urinary catheters are very often infected with bacteria which often can be very uncomfortable or even painful to the patients and which may impair the perceived quality of life of the patient. By using the delivery device catheter of the invention and a drug against the bacteria as a chemical compound in the inflation liquid, the infection may be avoided or treated during use of the catheter.

In an embodiment the balloon is a compliant balloon which continuously expands upon filling fluid into the balloon e.g. upon increasing the pressure within the balloon.

In an embodiment the balloon is a non-compliant balloon which expands until a specific size or size range is reached upon filling fluid into the balloon, e.g. upon increasing the pressure within the balloon. Such type of balloon is for example described in WO 2010/042869. In an embodiment the delivery device of the invention is a catheter as described in WO 2010/042869 with the difference that at least a section of the balloon wall is a delivery membrane as described herein.

In an embodiment the balloon is a folded balloon which unfolds upon filling fluid into the balloon.

In an embodiment the catheter comprises a diagnostic probe, such a probe comprising a marker such as a fluorescence marker. The probe is preferably adapted for detection of at least one biomarker such as, but not limited to, pH value, temperature, moisture level or infection level and combinations thereof, e.g. by multiplexing technique.

In an embodiment the diagnostic probe is configured for changing upon a change of the at least one biomarker. The change of the probe is preferably optically readable. The change may for example be a change of colour. Suitable probes can be found in “The Molecular Probes® Handbook—A Guide to Fluorescent Probes and Labelling Technologies”, freely available on the Internet:

https://www.lifetechnologies.com/dk/en/home/references/molecular-probes-the-handbook.html

In an embodiment the probe is incorporated or mounted in a distal end of the catheter, such as in a distal tip of the catheter, such that it will be positioned in the bladder of a patient during use. If an infection is developing the infection can be detected by the probe. The probe can for example be read out optically or electrically e.g. using an electrochemical reader. For electrochemical read-out the catheter may comprise one or more incorporated electrical wires or strips such that the electrical potential of the probe can be read out. This may be in the form of impedance read-out.

In an embodiment the probe can be observed externally or internally to the membrane e.g. visually or optically e.g. where the probe comprises a fluorescent marker and/or an enzymatic marker.

In an embodiment the catheter comprises a read-out structure for reading the probe and transmitting the read signal to a displaying element for visually or audibly displaying, wherein the read-out structure comprises an optical or electrochemical reader.

A read-out structure e.g. based on optical read-out of for example a fluorescence based signal or based on electrochemical read-out, will advantageously report the infection level and denote the type of microorganism. Depending on the type of microorganism one or more drugs can be introduced in the inflation liquid for the balloon for local treatment of the infection.

In an embodiment the catheter comprises an optical read-out structure. The catheter comprises a drainage tube and a distal tip. The drainage tube comprises a channel for draining urine. The channel extends to the tip, and the tip comprises or carries the probe. The channel is configured for functioning as a waveguide, e.g. when filled with liquid, such as urine or water. The probe is advantageously positioned such that at least a part of a light beam fed to the channel reaches the probe, such that reflected or scattered light can be detected by an optical reader. Thereby the channel forms a light beam path for the detection.

In an embodiment the catheter comprises an optical read-out structure. The catheter comprises an inflation tube and a distal tip. The inflation tube comprises a channel for inflating the balloon. The channel extends to the tip, and the tip comprises or carries the probe. The channel is configured for functioning as a waveguide e.g. when filled with liquid, such as buffer or water. The probe is advantageously positioned such that at least a part of a light beam fed to the channel reaches the probe, such that reflected or scattered light can be detected by an optical reader. Thereby the channel forms a light beam path for the detection.

In one embodiment the inflation channel guides light to heat the balloon via gold particles or other heating mechanisms to increase the release of drug. The guiding of light for heating the balloon may be coupled to the sensor in a feed-back loop.

In an embodiment the catheter comprises an optical read-out structure, wherein the catheter comprises a drainage tube and/or an inflating tube and a distal tip. The drainage tube comprises a channel for draining urine, and the inflation comprises a channel for inflating the balloon. The at least one of the channels extends to the tip and the probe is fixed on an outer surface of the tube, wherein the tube has a transparent window to the probe. The at least one channel is configured for functioning as a waveguide e.g. when filled with liquid, such as urine or water for reading scattered light from the probe. In an embodiment the probe is mounted on the balloon and the inflation lumen is applied as an optical waveguide.

In an embodiment the delivery device is a ring-shaped delivery device and the cavity is ring-shaped or semi-ring-shaped within the ring-shaped delivery device. The ring shaped delivery device may in principle be used for delivery of any type of drug.

In an embodiment the delivery device is a vaginal ring preferably adapted to be positioned to surround the cervix of a female mammal, such as a human or mammal, such as a human or an animal e.g. a horse, a dog or a cat.

In an embodiment the delivery device is a contraceptive delivery device such as a vaginal ring (P-ring) containing a drug for preventing pregnancy optionally in combination with one or more other drugs, such as in the form of a drug combination for preventing HIV and/or pregnancy.

In an embodiment the delivery device is a contraceptive delivery device for a mammal, such as a human or a cat.

Such contraceptive delivery device for a cat may for example be used in replacement of spaying or neutering the cat, thus the contraceptive delivery device is much gentler to the cat and complications which are often linked to spaying or neutering by surgery may be avoided.

In an embodiment the delivery device contains in its cavity a drug for treating symptoms of alzheimers, such as T3D-959, donepezil, galantamine, memantine, rivastigmine or any combinations thereof.

In an embodiment the delivery device contains in its cavity a substance comprising drug(s) selected from Abomorphin, Hormone replacement, Prograstrorel, cyproterone and combinations thereof.

In an embodiment the delivery device is a contraceptive device e.g. shaped as a ring and the cavity is formed as a ring or a semi-ring within the device. Advantageously the delivery device is a contraceptive device shaped as a hollow ring. In an embodiment the hollow ring consists entirely of the delivery membrane forming the cavity into which the chemical substance can be injected e.g. using an injection needle as described above. In an embodiment the entire hollow ring is formed by the delivery membrane and the chemical substance, where the delivery membrane forms the cavity comprising the chemical substance in dry or liquid e.g. dissolved or partly dissolved form.

In an embodiment the delivery device is a P-ring (vaginal ring) containing a drug combination for preventing HIV or other infections and/or pregnancy. Advantageously the P-ring is not expandable but retains its shape during use. In an embodiment the P-ring cavity comprises a hydrogel forming a reservoir for the substance to be delivered by the delivery device in form of a P-ring.

In an embodiment the delivery device is a capsule, such as a capsule comprising the delivery membrane which is defining the cavity, and where the chemical substance is or is to be injected into the cavity. In an embodiment the delivery device is a capsule having a volume of at least about 0.01cm³, such as a volume of at least about 0.1 cm³.

Advantageously the capsule having a volume of at least about 0.05 cm³, such as a volume of at least about 0.1 cm³, such as a volume of at least about 0.5 cm³, such as a volume of at least about 1 cm³, such as a volume of at least about 2 cm³, such as a volume of at least about 5 cm³. Thereby the capsule may be loaded with chemical substance using a needle.

In an embodiment the capsule is shaped as a sphere. Alternatively the capsule may have other shapes, such as oval or egg-shaped.

In an embodiment the capsule is oblong, egg shaped, oval and/or flattened. The term “flattened” means that the capsule has a shape with two opposite substantially flat outer surfaces which are is substantially parallel planes.

Advantageously the capsule has a smallest diameter of at least about 5 mm, such as at least about 1 cm, such as at least about 2 cm.

In an embodiment the capsule comprises drug trapped in liposomes which are dispersed in a polar liquid as described above. Such capsule may e.g. be implanted into a mammal for controlled drug release. The drug trapped in liposomes which are dispersed in a polar liquid may be replaced at preselected intervals or when needed. The drug may e.g. be a pain killer which the patient can trigger to be released.

In an embodiment the drug is Rivastigmine (sold under the trade name Exelon) which is a parasympathomimetic or cholinergic agent for the treatment of symptoms of mild to moderate dementia of the Alzheimer's type and dementia due to Parkinson's disease. The drug can be administered orally or via a transdermal patch; the latter form reduces the prevalence of side effects which typically include nausea and vomiting. The drug is eliminated through the urine, and appears to have relatively few drug-drug interactions.

In an embodiment the capsule is adapted for use in waste water treatment or in fuel-cells.

The invention also relates to a delivery device suitable for delivering a chemical substance comprising one or more chemical compounds, the delivery device comprises a closed cavity comprising the chemical substance, the cavity is defined by an innermost wall surface, wherein at least a section of the inner wall surface constitutes an inner surface of a delivery membrane wherein the delivery membrane comprises an interpenetrating polymer network substrate comprising a host polymer and a guest polymer, where the guest polymer is interpenetrating the host polymer to form substantially continuous pathways within the host polymer.

Preferably the delivery device is as described above wherein the chemical substance is contained in the closed cavity.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

All features of the invention including ranges and preferred ranges can be combined in various ways within the scope of the invention, unless there are specific reasons for not combining such features.

### BRIEF DESCRIPTION OF DRAWINGS AND EXAMPLE

The invention will be explained in more detail below in connection with a preferred embodiment and with reference to the drawings in which:

FIG. 1 is a chart of the mass in mg that migrates through the balloons over time in days of the IPN sample and the blind prior art sample of example 1.

FIG. 2 shows an embodiment of a delivery device of the invention in the form of a balloon catheter.

FIG. 3 is a schematic diagram of use showing an embodiment of a balloon catheter of the invention.

FIG. 4 is a schematic drawing of the human female reproductive system comprising the uterus and with an embodiment of the p-ring of the invention mounted to surround the cervix.

FIG. 5 is a schematic side view of the human female reproduction system corresponding to the drawing of FIG. 4.

FIG. 6 illustrates a capsule of an embodiment of the invention comprising a chemical substance.

FIG. 7a is a cryo-SEM image of a silicone host polymer.

FIG. 7b is a corresponding illustration of the silicone host polymer of FIG. 7a.

FIG. 8a is a cryo-SEM image of the silicone host polymer with interpenetrating guest polymer with a structure comprising a plurality of beads.

FIG. 8b is a corresponding illustration of the silicone host polymer/guest polymer IPN of FIG.8.

FIG. 9a is a SEM image of the silicone host/guest polymer IPN of FIGS. 8a and 8b which has been swollen with water.

FIG. 9b is a corresponding illustration of water swollen the silicone host polymer/guest polymer IPN of FIG. 9a.

FIG. 10 comprises the illustrations of FIGS. 7b, 8b, 9b side by side.

FIG. 11 is a SEM image of a silicone host polymer before being subjected to residual extraction.

FIG. 12 is a SEM image of the dry silicone host/guest polymer IPN of FIG. 9a.

FIG. 13 illustrates a capsule of an embodiment of the invention comprising a chemical substance comprising liposomes.

The figures are schematic and may be simplified for clarity. Throughout, the same reference numerals are used for identical or corresponding parts.

### EXAMPLE 1

Diffusion of Methylene Blue in Foley Catheters

Purpose: An experiment was designed to qualitatively and quantitatively measure the diffusivity of methylene blue through the retention balloon of a urinary Foley catheter and to calculate the diffusion coefficient, D, for a catheter according to an embodiment of the invention and a prior art catheter.

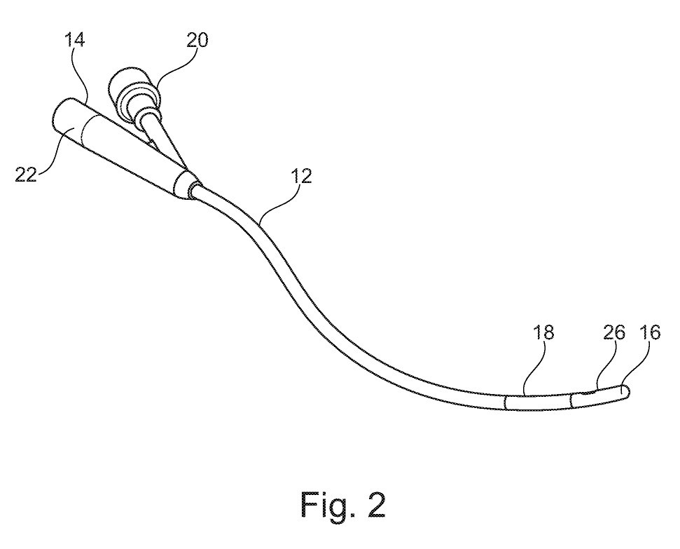

Materials and Methods: Two catheters were obtained from Bactiguard. One of the catheters was treated by removing the balloon and replacing it with an IPN of silicone as host polymer and hydrogel as guest polymer where the hydrogel content was 24% (called the IPN catheter). The other catheter was untreated (called the prior art catheter). The IPN catheter and the prior art catheter were inflated with aqueous glycol solution to check for any leaking. After rinsing and cleaning them, they were each filled with 10 ml 10 mg/mL aqueous methylene blue solution and each catheter was partly (including the tip (16), hole for drainage (26), balloon (18) and ⅔ of the shaft (12) on FIG. 2) submerged into 1 L distilled water with stirring at 200 rpm at ambient temperature. The volumes were kept constant during the experiment. The concentration of methylene blue in the 1 L surrounding water was periodically measured by applying a Thermo Scientific Evolution 220 UV-vis spectrophotometer at 664 nm.

Results: The experiment showed that methylene blue migrates through the balloon material on the IPN catheter turning the surrounding liquid blue. Over the period of the experiment (24 days) it was not possible to detect methylene blue in the surrounding liquid of the prior art catheter. This strongly indicates that methylene blue cannot diffuse through the prior art balloon, but the IPN treatment (current invention) enables diffusion.

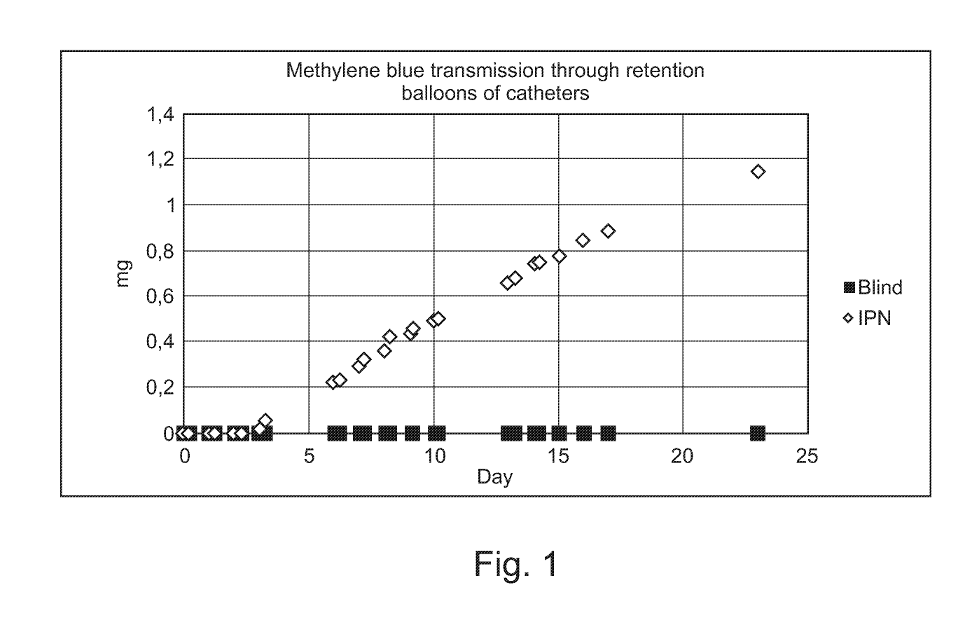

FIG. 1 is a chart of the mass in mg that diffuses through the balloons over time in days of the IPN catheter and the blind prior art catheter (blind sample).

The blind sample showed no diffusion into the solvent whereas the IPN catheter had a lag-phase of 3 days before methylene blue could be detected in the solvent.

The diffusion coefficients, D, of methylene blue in IPN catheter and blind sample can be estimated by the equation (1).

$$D = \frac{Q \cdot X}{Δ c \cdot A \cdot t} \qquad ( 1 )$$

Where Q is the total amount of methylene blue (permeant) that has passed through an area, A, during time, t. X is the thickness of the balloon and Δc is the difference in concentration between the outer surface and the inner surface of the balloon.

Qcan be obtained by determining the linear regression for the IPN after the lag-phase has ended i.e. from day 3 to day 14 on FIG. 1. The linear regression is given by equation (2)

m=0.061·t−0.1236 (2)

Q is the slope of the linear regression, i.e. 0.061 mg per day.

It is not possible to measure the thickness of the balloon, X when the balloon is inflated. However, X can be estimated by assuming that the volume of the balloon material does not change when the balloon is inflated. In the inflated condition the shape of the balloon can be approximated by a spherical shell and in the un-inflated (relaxed) condition the shape of the balloon can be approximated with a hollow cylinder. The volume of the balloon material can be calculated from the relaxed condition and can then be used to calculate the thickness of the balloon in the inflated condition.

The volume of a hollow cylinder, V_{hc}, is given by equation (3).

V_{hc}=h·π·(r₁²−r₂²) (3)

Where h (2.50 cm) is the height of the cylinder and r₁(0.365 cm) and r₂(0.315 cm) is the outer and inner radii respectively. This yields a total volume of the balloon material of V_{hc}=0.2670 cm³.

V_{hc}=2.50 cm·π·((0.365 cm)²−(0.315 cm)²)=0.2670 cm³ (4)

As the cavity between the shaft and the balloon is filled with the inflation liquid, the balloon material will assume the shape of a spherical shell. However, the volume of the balloon material will still be Vₛₛ=V_{hc}=0.2670 cm³. The volume of a spherical shell, Vₛₛ, is given by equation (5).

$${V}_{ss} = \frac{4}{3} \cdot π \cdot ( {r}_{1}^{3} - {r}_{2}^{3} ) \qquad ( 5 )$$

The thickness of the balloon material in the inflated state, X is given by equation (6).

X=r₁−r₂ (6)

r₁ can be measured to 1.25 cm. The inner radius, r₂, can be calculated by isolating r₂ in equation (5). This is given by equation (7).

$${r}_{2} = \sqrt[3]{{r}_{1}^{3} - \frac{3 \cdot {V}_{ss}}{4 \cdot π}} - \sqrt[3]{{( 1.25 cm )}^{3} - \frac{3 \cdot 0.2670 cm}{4 \cdot π}} = 1.2362 cm \qquad ( 7 )$$

Xcan now be calculated from equation 6. This is given by equation (8).

X=1.25 cm−1.2362 cm=0.0138 cm (8)

Now, we have values for the parameters in the numerator in equation (1) and we need to find values for the denominator.

Δc is the concentration difference between the outer surface and the inner surface of the balloon material. As the initial concentration of methylene blue is 10 mg/ml and the cavity between the shaft and the balloon material is filled with 10 ml, there is 100 mg methylene blue in the cavity. As can be seen on FIG. 1 about 1 mg methylene blue is migrated though the balloon material over a period of 20 days. This is only 1%. It is therefore safe to assume that the concentration at the inner surface is 10 mg/ml throughout the experiment. As there is constant mixing on the outside of the catheter the concentration can be assumed to be homogenously distributed. Again, 1 mg methylene blue in 1 L=1000 ml water is 0.001 mg/ml=0 mg/ml. Therefore Δc is about 10 mg/ml throughout the experiment.

A is the surface area of the balloon material in the inflated condition. The area of a sphere with radius r=1.25 cm is given by equation (9).

A=4·π·r²=4·π·(1.25 cm)²=19.63 cm² (9)

now, we can estimate the diffusion coefficient of methylene blue though the balloon material by inserting in equation (1). This is given by equation (10).

$$D = \frac{Q \cdot X}{Δ c \cdot A \cdot t} = \frac{0.061 mg \cdot 0.0138 cm}{10 mg / {cm}^{3} \cdot 19.63 {cm}^{2} \cdot 86400 s} = 4.95 \cdot {10}^{- 11} {cm}^{2} / s \qquad ( 10 )$$

As it was not possible to detect any methylene blue in the surrounding liquid of the prior art catheter throughout the experiment the diffusion coefficient is D=0 cm²/s.

Conclusions: Methylene blue diffused from the cavity through the balloon material on the IPN catheter into the solvent, while no diffusion with the prior art catheter was observed. The diffusion coefficient for the delivery membrane of the IPN catheter was calculated to be 4.95·10⁻¹¹ cm^{2/}s.

The balloon catheter shown in FIG. 2 is of the Foley type and comprises a catheter body 12 with a proximal end 14 and a distal end 16. The catheter also includes a balloon 18, an inflation lumen 20, a drainage lumen 22, and an adapter 24.

The balloon 18 is deflated for insertion into a patient. The balloon 18 is disposed near the distal end 16. The inflation lumen 20 extends within the catheter body 12 from the proximal end 14 to the balloon 18, in fluid communication with the balloon 18, for inflating and deflating the balloon 18.

The catheter drainage lumen 22 extends from the proximal end 14 to the distal end 16. The distal end 16 includes an opening 26 in fluid communication with the drainage lumen 22 to facilitate drainage of urine from the bladder of a patient.

In the shown embodiment the balloon 18 is the delivery membrane, which can be inflated e.g. with a fluid comprising chemical substances for being delivered through the delivery membrane as discussed above.

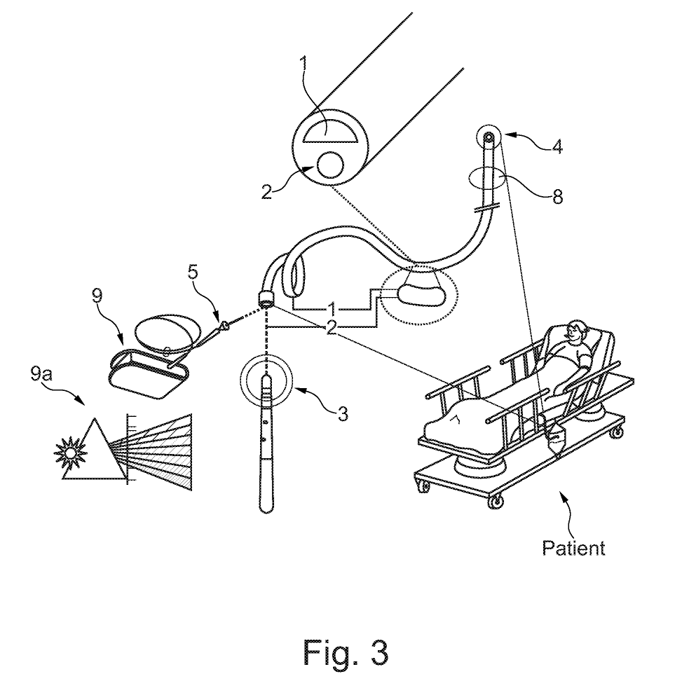

FIG. 3 is a schematic diagram of use another embodiment of a balloon catheter of the invention.

The balloon catheter shown in FIG. 3 comprises a catheter body 12 with a proximal end and a distal end with a probe 4. The catheter also includes a balloon 8 expanded with a fluid comprising chemical substances to be delivered through the balloon wall which constitutes the delivery membrane. The catheter further comprises an inflation lumen 2, a drainage lumen 1. A cut-out section of the body illustrates that the inflation lumen 2 and the drainage lumen 1 extend along the body.

As illustrated in FIG. 3 the balloon catheter is used by a patient.

The probe 4 is advantageously as described above, e.g. a probe with an optical biomarker.

The inflation lumen is configured for functioning as a waveguide e.g. when filled with liquid. For reading out a laser 3 is arranged to transmit a beam via the inflation lumen. The probe 4 is positioned such that at least a part of a light beam fed to the channel (inflation lumen) 2 reaches the probe 4, such that reflected or scattered light can be detected by an optical reader 9. And a readout spectrum can be obtained as illustrated with the spectrum 9a. Thereby the channel forms a light beam path for the detection.

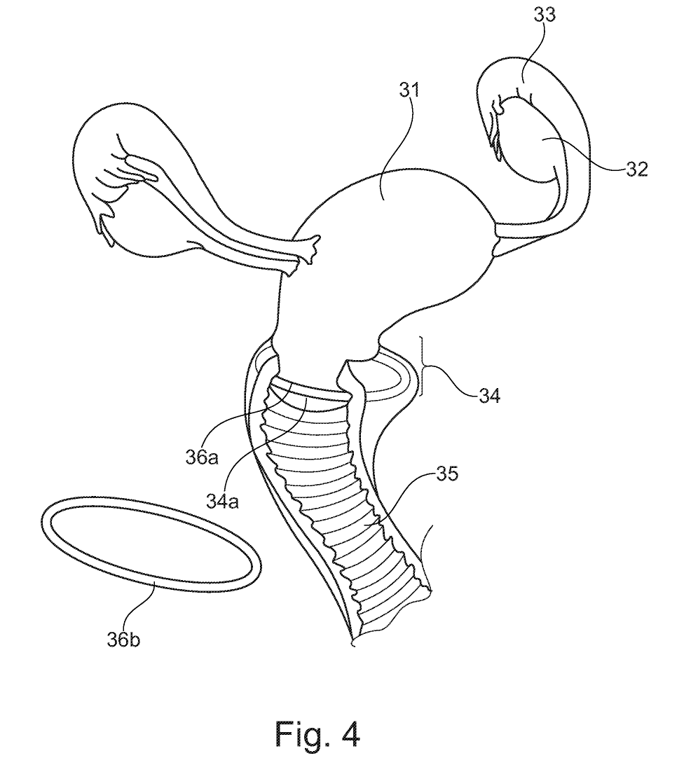

The human female reproductive system shown in FIG. 4 comprises the uterus 31, the fallopian tubes 32, the ovaries 33 and the cervix 34. The lower part of the cervix 34a leads into the vagina 35 and the P-ring 36a is mounted in the vagina to surround the lower part of the cervix 34a. Reference 36b illustrated the p-ring prior to mounting.

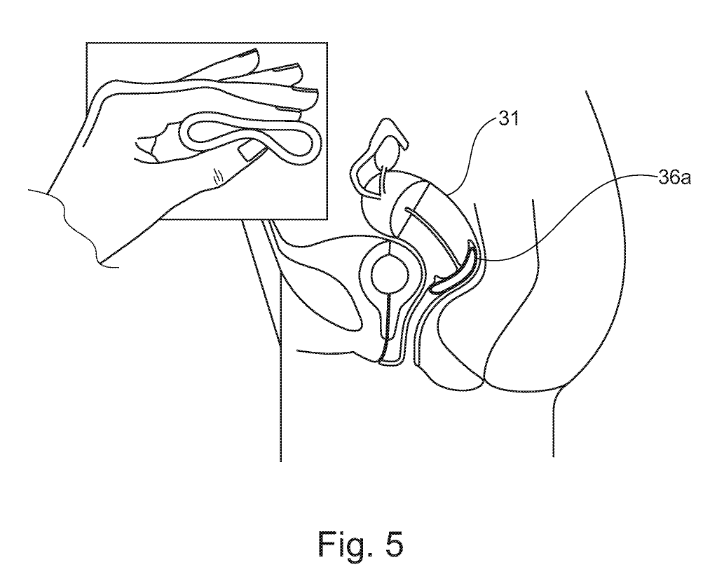

FIG. 5 is a schematic side view of the human female reproduction system corresponding to the drawing of FIG.4. It is illustrated that the P-ring is very flexible and is easily bendable by hand.

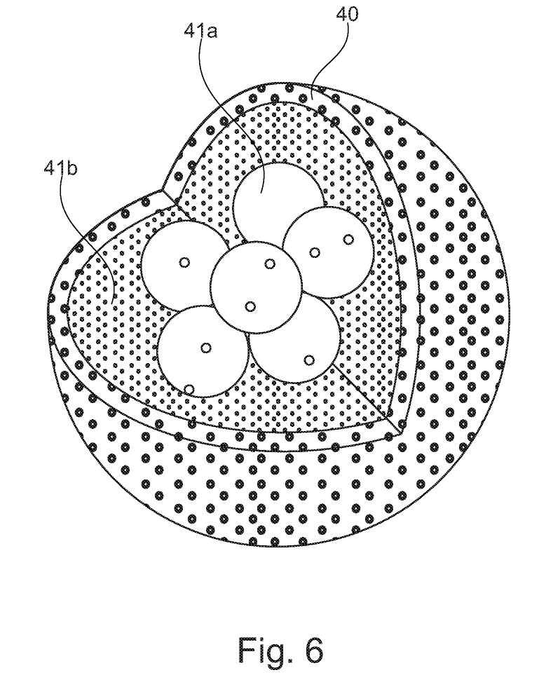

The capsule shown in FIG. 6 comprises a capsule wall 40 constituting the delivery membrane and forming a cavity comprising the chemical substances 41a in a fluid 41. In the shown embodiment a section of the capsule is cut out for illustrative purposes. In an alternative embodiment the whole cavity encapsulated by the capsule wall 40 is filled with a chemical substance in dry form.

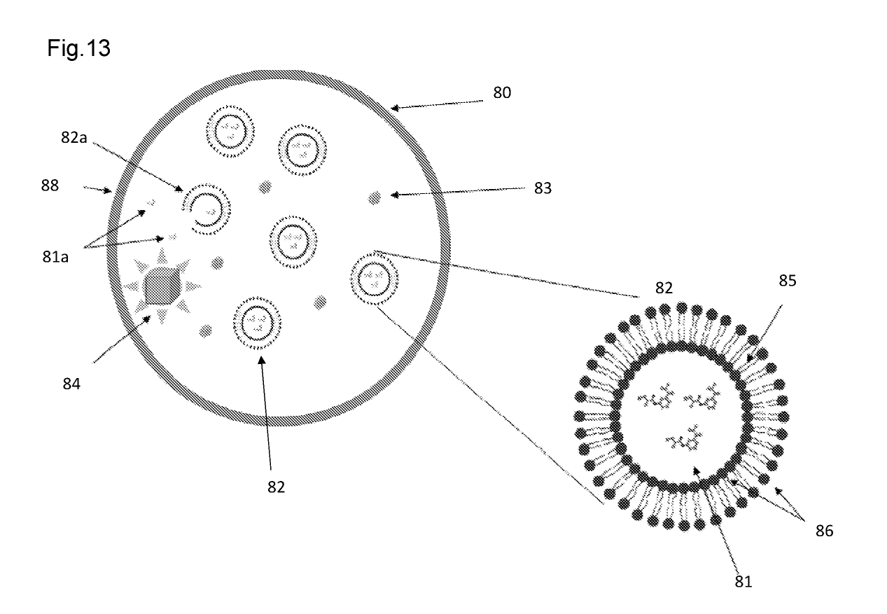

The capsule of FIG. 13 comprises a capsule wall 80 constituting the delivery membrane and forming a cavity comprising the chemical substances. The chemical substance comprises drug 81 trapped in liposomes 82 together with gold nanoparticles 83, and one or more light emitting units 84, such as quantum dot(s) or micro light-emitting diode(s).

One of the Liposomes is illustrated in enlarged view and here it can be seen that the liposome 82 comprises a lipid bilayer 85 with polar ends 86 turning inwards and outwards.

The drug 81, 81a is preferably Rivastigmine.

The light emitting unit 84 may advantageously be triggered from external impact e.g. as described above. When the light emitting unit 84 emits light it will be absorbed by the gold particles 83, and the temperature of the gold particles 83 will increasing. When the liposomes reaches a certain temperature they rupture as shown with the ruptured liposome 81a and the drug will be released from the liposome. The released drug may now freely migrate across the delivery membrane 80 membrane to the target spot for the drug, e.g. if implanted in the brain to the exposed brain tissue. Rivastigmine can thereby be released to the brain tissue with a desired release profile. When the amount of liposomes are reduced or there is no more left the substance within the capsule 88 may be withdrawn using a needle and it may be replaced with fresh substance.

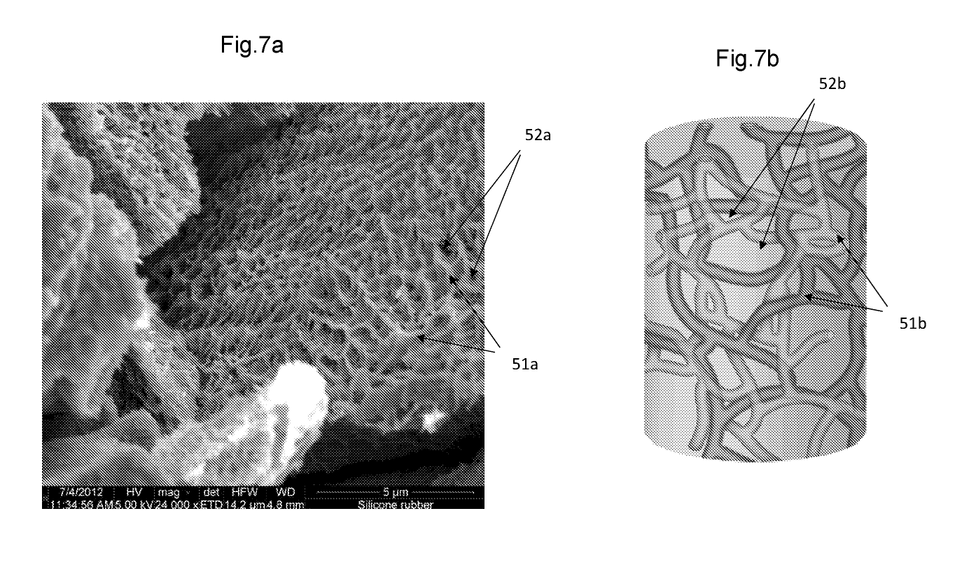

The silicone host polymer shown in FIG. 7a is a PDMS silicone polymer cooled in liquid nitrogen (T˜−196° C.) and removed from the nitrogen immediately prior to acquiring the cryo-sem image. The morphology/structure of PDMS have been frozen by placing the sample in liquid nitrogen, which has a temperature below the glass transition temperature of silicone (Tg=−125° C.). It can be seen that the host polymer has a structure of a network of strand shaped filaments 51a and comprises a plurality of intrastrand pathways 52a. The strand shaped filaments are substantially flat.

In FIG. 7b the same host polymer is illustrated with the network of strand shaped filaments 51b and the intrastrand pathways 52b.

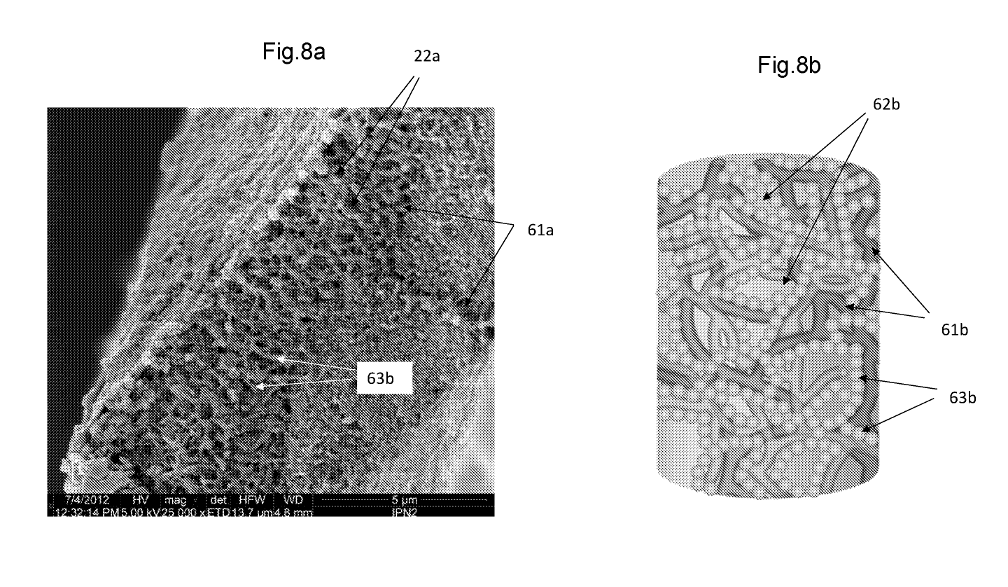

FIG. 8a show the same silicone host polymer with interpenetrating guest polymer also in liquid nitrogen (T˜−196° C.) with a structure comprising a plurality of beads 63a forming a substantially continuous pathways within the host polymer. The guest polymer has been loaded into the host polymer using CO₂ in dense gas state as solvent. It can be seen that the plurality of beads 63a of guest polymers are arranged along the strand shaped filaments 61a to form the substantially continuous pathways within the host polymer. It can be seen that the intrastrand pathways 62a are only partly filled. It is believed that this has the effect of making the final IPM delivery membrane very pliable and soft.

It can be seen that the guest polymer has affinity to the host polymer.

FIG. 8b illustrates the silicone host polymer with interpenetrating guest polymer of FIG. 8a and it can be seen more clearly that the beads 63b of guest polymers are arranged along the strand shaped filaments 61b to form the substantially continuous pathways within the host polymer and that the intrastrand pathways 62b are only partly filled.

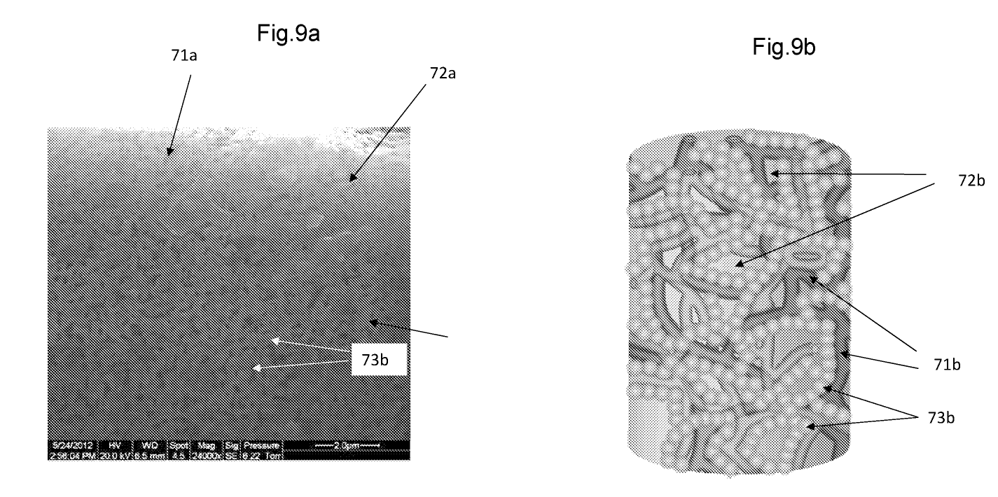

In FIG. 9a the silicone host/guest polymer IPN of FIGS. 8a and 8b which has been swollen with water at room temperature. The strand shaped filaments 71a are almost fully covered with the beads 73a which have swollen and thereby increased in size. In the SEM image the intrastrand pathways 72a can only slightly be seen. In the corresponding illustration in FIG. 9b it can be seen that the guest polymer beads 73b has been swollen. For illustrative purpose the swollen guest polymer beads are illustrated in less swollen state than in the real life cry-SEM image of FIG. 9a.

However, it can be seen that the swollen guest polymer beads takes up more of the intrastrand pathways 72b between the strand shaped filaments 71b.

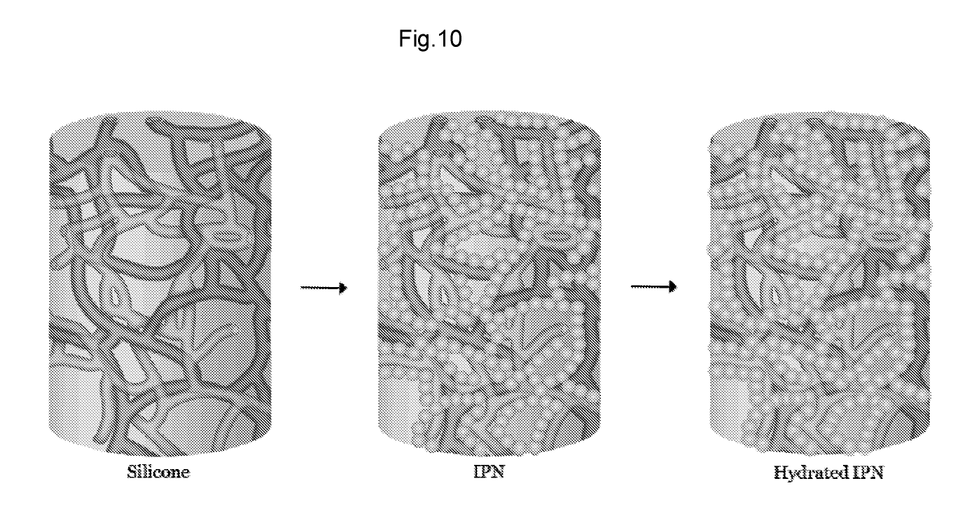

In FIG. 10 the illustrations of FIGS. 7b, 8b, and 9b are shown side by side and it can be seen how the host alone differs from the silicone host/guest polymer IPN and further the changes when the silicone host/guest polymer IPN is swollen with water.

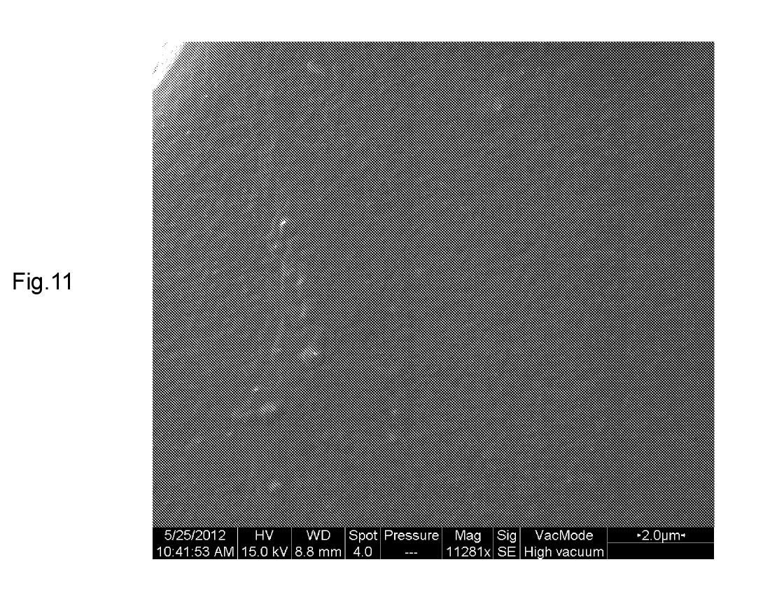

FIG. 11 shows a SEM picture the PDMS silicone polymer at room temperature. There is no visible network of strand shaped filaments and intrastrand pathways, because the temperature is much higher than the glass transition temperature of silicone (Tg=−125° C.).

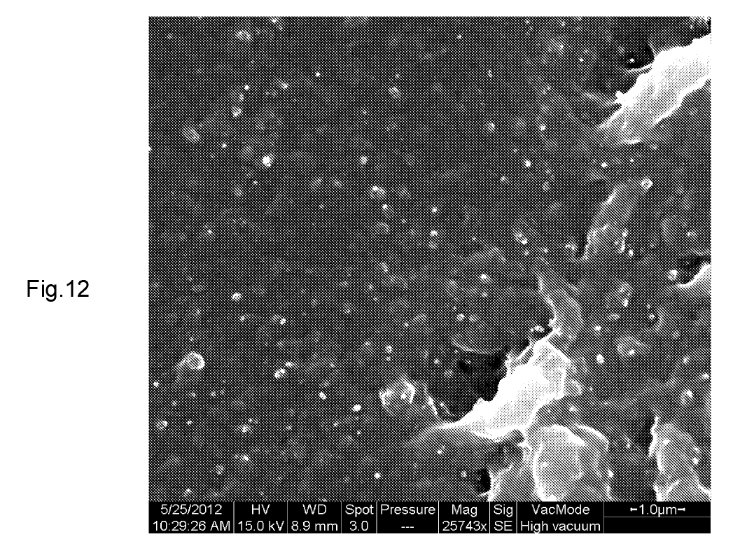

In FIG. 12 a SEM image of the dry silicone host/guest polymer IPN at room temperature of FIG. 9a is shown and it can be seen that the surface as a structure comprising a plurality of bead like structures. The guest polymer (hydrogel) has a Tg of 100° C. and is solid at room temperature. Therefore the hydrogel takes the shape as solid beads. The host polymer (silicone) has a Tg of −125° C. and therefore behaves as a liquid at room temperature and embeds the solid hydrogel beads.

Although embodiments have been described and shown in detail, the invention is not restricted to these, but may also be embodied in other ways within the scope of the subject matter defined in the following claims. In particular, it is to be understood that other embodiments may be utilized and structural and functional modifications may be made without departing from the scope of the present invention.

1. A delivery device suitable for delivering a chemical substance comprising one or more chemical compounds, the delivery device comprises a closed cavity, the cavity is defined by an innermost wall surface, wherein at least a section of the inner wall surface constitutes an inner surface of a delivery membrane wherein the delivery membrane comprises an interpenetrating polymer network substrate comprising a host polymer and a guest polymer, where the guest polymer is interpenetrating the host polymer to form substantially continuous pathways within said host polymer.

2. The delivery device of claim 1, wherein said delivery membrane has an outer surface opposite to said inner surface of said delivery membrane, said substantially continuous pathways of said guest polymer within said host polymer extend from the inner surface to the outer surface of said delivery membrane.

3. The delivery device of claim 1 or claim 2, wherein at least a section of said delivery membrane has a thickness of from 0.1 μm-10 cm, such as from 1 μm to 1 cm preferably from 10 μm to 1 mm.

4. The delivery device of any one of the preceding claims, wherein the host polymer is a cross-linked polymer such as a cross-linked elastomer, e.g. thermoplastic elastomer (TPE), polyolefin elastomer (POE), polyurethane (PU), rubber, e.g. latex rubber, silicone or any combinations thereof.

5. The delivery device of any one of the preceding claims, wherein the host polymer is physically cross-linked; the host polymer is preferably a physically cross-linked TPE.

6. The delivery device of any one of the preceding claims, wherein the host polymer is chemically cross-linked; the host polymer is preferably covalently cross-linked.

7. The delivery device of any one of the preceding claims, wherein the host polymer is an elastomer, preferably selected from thermoplastic elastomers (TPEs) polyolefin elastomers (POEs), thermoplastic polyurethane (TPU), rubber, e.g. latex rubber, silicones and/or any combinations thereof.

8. The delivery device of claim 7, wherein the host polymer comprises thermoplastic polyurethane (TPU).

9. The delivery device of claim 7, wherein the host polymer comprises silicone.

10. The delivery device of any one of the preceding claims, wherein the host polymer has a structure of a network of strand shaped filaments comprising a plurality of intrastrand pathways.

11. The delivery device of claim 10, wherein the polymer has a structure of a network of substantially flat strand shaped filaments.

12. The delivery device of claim 10 or claim 11, wherein the intrastrand pathways has a volume of at least about 30% of the total volume of the delivery membrane, such as at least about 40%, such as at least about 50%, such as at least about 60% of the total volume of the delivery membrane.

13. The delivery device of any one of the preceding claims, wherein the guest polymer comprises a gel selected from hydrogel, aerogel, xerogel or any combinations thereof.

14. The delivery device of any one of the preceding claims, wherein the guest polymer comprises a homopolymer, preferably polymerized from an acrylate monomer or a vinyl polymer, more preferably n-vinyl pyrolidone (nVP), styrene; oxygen-, phenyl, amino and nitrogen-containing acrylic and methacrylic derivatives, e.g. acrylic esters, acrylic acids, methacrylic acid and -esters, alkyl and hydroxyalkyl acrylates and methacrylates; functionalized methacrylates such as 2-hydroxyethyl methacrylate (HEMA), glycerol monomethacrylate (GMMA), heptaflurobutyl acrylate (HFBA), 2-methacryloyloxyethyl phosphorylcholine (MPC) and [2-(methacryloyloxy)ethyl]-dimethyl-(3-sulfopropyl)-ammonium hydroxide (Betain); alkyl substituted acrylates and methacrylates such as methyl methacrylate (MMA), ethyl methacrylate (EMA), butyl methacrylate (BMA), dodecyl methacrylate (DMA); PEGylated (meth)actylates such as poly(ethylene glycol) methyl ether methacrylate (PEGMEMA) and poly(ethylene glycol) methyl ether acrylate (PEGMEA); substituted 62 - and γ-lactones, lactic acid monomers; carbohydrides and fluorinated monomers; urethanes; mono- and di-functional alcohols; carboxylic acids; amines; isocyanates; epoxides; aromatics carrying alkyl group(s); sulfonated aromatics, aromatic resins; imidazole; imidazol derivatives; zwitterionic monomers; pyrazoles; quaternary ammonium monomers and any combinations thereof.

15. The delivery device of any one of the preceding claims 1-13, wherein the guest polymer comprises a copolymer, preferably polymerised from monomers comprising silanes, e.g. tetraethylorthosilicate or tetraethoxysilane (TEOS), an acrylate monomer or a vinyl polymer, more preferably n-vinyl pyrolidone (nVP), styrene; oxygen-, phenyl, amino and nitrogen-containing acrylic and methacrylic derivatives, e.g. acrylic esters, acrylic acids, methacrylic acid and -esters, alkyl and hydroxyalkyl acrylates and methacrylates; functionalized methacrylates such as 2-hydroxyethyl methacrylate (HEMA), glycerol monomethacrylate (GMMA), heptaflurobutyl acrylate (HFBA), 2-methacryloyloxyethyl phosphorylcholine (MPC) and [2-(methacryloyloxy)ethyl]-dimethyl-(3-sulfopropyl)-ammonium hydroxide (Betain); alkyl substituted acrylates and methacrylates such as methyl methacrylate (MMA), ethyl methacrylate (EMA), butyl methacrylate (BMA), dodecyl methacrylate (DMA); PEGylated (meth)actylates such as poly(ethylene glycol) methyl ether methacrylate (PEGMEMA) and poly(ethylene glycol) methyl ether acrylate (PEGMEA); substituted β- and γ-lactones, lactic acid monomers; carbohydrides and fluorinated monomers; urethanes; mono- and di-functional alcohols; carboxylic acids; amines; isocyanates; epoxides; aromatics carrying alkyl group(s); sulfonated aromatics: aromatic resins; imidazole; imidazol derivatives; zwitterionic monomers; pyrazoles; quaternary ammonium monomers and any combinations thereof.

16. The delivery device of claim 13, wherein the guest polymer comprises poly(2-hydroxyethyl methacryate) (PHEMA), preferably the gel is a copolymer of poly(2-hydroxyethyl methacryate) (PHEMA) and PEGMEA.

17. The delivery device of any one of the preceding claims, wherein the guest polymer has a max. water swelling at 25° C. of about 10-10000% by mass of its dry mass, such as of about 10-5000% by mass of its dry mass, such as of about 10-500% by mass of its dry mass.

18. The delivery device of any one of the preceding claims, wherein the guest polymer has a max. water swelling at 25° C. w/w of its dry mass which is higher than the max. water swelling at 25° C. w/w of the host polymer.

19. The delivery device of any one of the preceding claims, wherein the delivery membrane has a max. water swelling at 25° C. of about 5-5000% by mass of its dry mass, such as of about 10-1000% by mass of its dry mass, such as of about 10-500% by mass of its dry mass, such as of about 10-40% by mass of its dry mass.

20. The delivery device of any one of the preceding claims, wherein the guest polymer has a structure comprising a plurality of beads forming said substantially continuous pathways within said host polymer.

21. The delivery device of claim 20, wherein the plurality of beads is arranged in side-by-side formations to form said substantially continuous pathways within said host polymer.

22. The delivery device of claim 20 or claim 21, wherein the host polymer has a structure of a network of strand shaped filaments comprising a plurality of intrastrand pathways and the plurality of beads of guest polymers are arranged along said strand shaped filaments to form said substantially continuous pathways within said host polymer.

23. The delivery device of claim 22, wherein the plurality of beads of guest polymers is adhered to said strand shaped filaments to form said substantially continuous pathways within said host polymer.

24. The delivery device of any one of the preceding claims, wherein the delivery membrane comprises a continuous matrix of the host polymer and a plurality of interconnected paths of the guest polymer, wherein the interconnected paths of the guest polymer preferably extend through the whole thickness of the membrane.

25. The delivery device of any one of the preceding claims, wherein the delivery membrane comprises a continuous matrix of the host polymer and a plurality of interconnected paths of the guest polymer, the delivery membrane has an outer surface and a plurality of paths of the guest polymer coincide with said inner surface and/or said outer surface.

26. The delivery device of any one of the preceding claims, wherein the delivery membrane comprises incorporation of one or more chemical compounds in the form of water soluble particles, such as drugs, buffers, surfactants, fragrances, dyes, flavours, antioxidants, nutrients, hormones, catalysts or any combinations thereof.

27. The delivery device of any one of the preceding claims, wherein the inner surface of said delivery membrane has a surface area of at least about 0.1 cm², such as at least about 1 cm², such as at least about 2 cm², such as at least about 5 cm², such as at least about 10 cm².

28. The delivery device of any one of the preceding claims, wherein the cavity and the inner surface of said delivery membrane is sufficiently large to load drug into said cavity via a loading lumen and/or a needle.

29. The delivery device of any one of the preceding claims, wherein the cavity is sufficiently large to load at least 50 μl, such as at least 100 pμ, such as at least 1 ml of fluid into said cavity.

30. The delivery device of any one of the preceding claims, wherein the cavity is an at least partially collapsed cavity.

31. The delivery device of any one of the preceding claims, wherein the wall comprising the innermost wall surface is inflatable.

32. The delivery device of any one of the preceding claims, wherein the cavity is or is designed to contain gas, preferably the wall comprising the innermost wall surface comprise a gas escape valve, such as a pressure relief valve.

33. The delivery device of any one of the preceding claims, wherein the delivery device is free of organic solvent other than ethanol.

34. The delivery device of any one of the preceding claims, wherein the cavity is adapted for being filled and/or refilled with one or more chemical compounds in the form of fluids and/or particles and/or particles dispersed or dissolved in a liquid, preferably the particles being selected from cells, catalysts, drugs, buffers, surfactants, fragrances, dyes, flavours or any combinations thereof, the cavity is preferably adapted for being filled with one or more chemical compounds by injecting via an inlet comprising a valve, such as an inflation lumen and/or by injection via the delivery membrane.

35. The delivery device of any one of the preceding claims, wherein the cavity comprises one or more chemical compounds in the form of fluids and/or particles, such as cells, catalysts, drugs, buffers, surfactants, fragrances, dyes, flavours or any combinations thereof.

36. The delivery device of any one of claims 26 to 35 wherein the chemical substance comprises a drug, preferably the drug is selected from anticonvulsants, analgesics, antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids sympathomimetics, parasympathomimetics, tranquilizers, urinary tract drugs, vaccines, vaginal drugs, vitamins, collagen, hyaluronic acid, nonsteroidal anti-inflammatory drugs, angiotensin converting enzymes, polynucleotides, polypeptides, polysaccharides, nicotine, adreanal hormones, painkillers, morphine, anticancer and combinations and mixtures thereof.

37. The delivery device of any one of claims 26 to 37 wherein the chemical substance comprises drug for treating symptoms of alzheimer's, such as T3D-959, donepezil, galantamine, memantine, rivastigmine or any combinations thereof.

38. The delivery device of any one of claims 26 to 37, wherein the chemical substance comprises a drug selected from components of the blood clotting cascade, such as factor VIII; birth control drugs, such as Levonorgestrel or ethinyl estradiol; anti-virus drugs (antiretroviral), such as anti-HIV drugs (dapivirine, tenofovir) or anti-hepatitis C (ribavirin).

39. The delivery device of any one of claims 26 to 38, wherein the chemical substance comprises a compound for human nutrition, such as a vitamin, a protein and/or a mineral or a component comprising one or more of the before mentioned.

40. The delivery device of any one of claims 26 to 39, wherein the chemical substance comprises a compound for microbiological nutrition, such as FeSO₄.7H₂O, ZnSO₄, KCI, MgSO₄, 7H₂O, NaNO₃, Glucose, NaCI, K₂HPO₄, NH₄H₂PO₄, CaC1₂.2H₂O, FeC1₃.6H₂O, NaH₂PO₄, Na₂HPO₄, NaHCO₃,(NH₄)₂ SO₄ or mixtures comprising any of the mentioned.

41. The delivery device of any one of claims 26 to 40, wherein the chemical substance comprises a fragrance compound, a flavour compound and/or a colour compound (e.g. pigment and dye).

42. The delivery device of any one of claims 26 to 41, wherein the chemical compound has a molar mass of up to about 300,000 g/mol., such as up to about 90,000 g/mol., such as up to about 50,000 g/mol., such as up to about 10,000 g/mol., such as up to about 5000 g/mol.

43. The delivery device of any one of claims 26 to 42, wherein the chemical compound comprises particles selected from stem cell(s), catalyst(s), nanoparticle(s) or combinations thereof.

44. The delivery device of any one of claims 26 to 43, wherein the cavity comprises particles comprising cells, such as stem cells or microorganisms, the cells are encapsulated in the cavity of the membrane and are preferably capable of producing at least one by-product which by-product is capable of migrating through the membrane, preferably via pathways of the guest polymer.

45. The delivery device of any one of claims 26 to 44, wherein the chemical substance comprises drug trapped in liposomes, preferably the liposomes are dispersed in a polar liquid, such as in an aqueous liquid.

46. The delivery device of claim 45, wherein the chemical substance comprises drug trapped in liposomes together with metal particles, preferably gold particles.

47. The delivery device of claim 45, wherein the chemical substance comprises drug trapped in liposomes together with metal particles and together with light emitting units which light emitting units can be triggered to emit light to heat the metal particles, preferably the metal particles are gold particles.

48. The delivery device of any one of the preceding claims, wherein the guest polymer is an aerogel or a xerogel of a hydrogel, preferably obtained by freeze-drying the hydrogel or by drying the hydrogel under supercritical or sub-supercritical conditions using a carrier gas such as CO₂, or by evaporation of solvents (e.g. water) at ambient conditions.

49. The delivery device of any one of claims 26 to 48, wherein the chemical component is selected relative to the membrane such that it migrates/through the membrane at zero order kinetics.

50. The delivery device of any one of the preceding claims, wherein the delivery device comprises a coating on at least a part of its inner or outer surface, preferably the coating is selected from an adhesive, a biopolymer and/or a hydrophilic coating, preferably at least a part the inner surface and/or the outer surface of the delivery membrane comprises a coating.

51. The delivery device of any one of the preceding claims, wherein the delivery device comprises a coating on at least a part of its inner and/or outer surface where the coating is of a material substantially identical to the guest polymer.

52. The delivery device of any one of the preceding claims, wherein the delivery device is a balloon catheter comprising at least one balloon forming the cavity.

53. The delivery device of claim 52, wherein the delivery device is a urinary catheter, such as a Foley catheter and the delivery membrane is the balloon or a section thereof, the balloon is adapted to being inflated using an inflation liquid comprising the chemical compound.

54. The delivery device of claim 52 or claim 53, wherein the balloon is a compliant balloon, which continuously expands upon filling fluid into the balloon.

55. The delivery device of claim 52 or claim 53, wherein the balloon is a non-compliant balloon, which expands until a specific size or size range is reached upon filling fluid into the balloon.

56. The delivery device of claim 52 or claim 53, wherein the balloon is a folded balloon, which unfolds upon filling fluid into the balloon.

57. The delivery device of any one of claims 52-56, wherein the catheter comprises a diagnostic probe, said probe is preferably adapted for detection of at least one biomarker such as, but not limited to, pH value, temperature, moisture level or infection level and combinations thereof (multiplexing).

58. The delivery device of claim 57, wherein the diagnostic probe is configured for changing upon a change of the at least one biomarker, said change of the probe is preferably optically readable.

59. The delivery device of claim 57 or 57, wherein said probe is incorporated or mounted in a distal end of the catheter, such as in a distal tip of the catheter.

60. The delivery device of any one of claims 57-59, wherein the catheter comprises a read-out structure for reading the probe and transmitting the read signal to a displaying element for visually or audibly displaying, wherein the read-out structure comprises an optical or electrochemical reader.

61. The delivery device of any one of claims 57-60, wherein the catheter comprises an optical read-out structure, wherein the catheter comprises an inflation and/or a drainage tube and a distal tip, said drainage tube comprises a channel for draining urine, said channel extending to the tip and said tip comprises or carries said probe, wherein said channel is configured for functioning as a waveguide e.g. when filled with liquid, such as urine or water.

62. The delivery device of any one of claims 57-60, wherein the catheter comprises an optical read-out structure, wherein the catheter comprises a drainage tube and a distal tip, said drainage tube comprises a channel for draining urine, said channel extends to the tip and said probe is fixed on an outer surface of the tube, wherein the tube has a transparent window to said probe, wherein said channel is configured for functioning as a waveguide, e.g. when filled with liquid, such as urine or water for reading scattered light from said probe.

63. The delivery device of any one of claims 1 to 51, wherein the delivery device is a ring-shaped delivery device and the cavity is a ring-shaped or semi-ring-shaped cavity within the ring-shaped delivery device.

64. The delivery device of claim 63 wherein the delivery device is a vaginal ring preferably adapted to be positioned to surround the cervix of a female.

65. The delivery device of claim 63 or claim 64, wherein the delivery device is a contraceptive delivery device such as a vaginal ring (P-ring) containing a for preventing drug pregnancy optionally in combination with one or more other drugs, such as in the form of a drug combination for preventing HIV and/or pregnancy.

66. The delivery device of any one of claims 63-65, wherein the delivery device is a contraceptive delivery device for a mammal, such as a human or an animal e.g. a horse, a dog or a cat.

67. The delivery device of claim 63 or claim 64, wherein the delivery device contains in its cavity a drug for treating symptoms of alzheimers, such as T3D-959, donepezil, galantamine, memantine, rivastigmine or any combinations thereof.

68. The delivery device of any one of claims 1 to 51, wherein the delivery device is a capsule, such as a capsule having a volume of at least about 0.01 cm³, such as a volume of at least about 0.1 cm³.

69. The delivery device of any one of claims 1 to 51, wherein the delivery device is a capsule, such as a capsule having a volume of at least about 0.5 cm³, such as a volume of at least about 1 cm³, such as a volume of at least about 2 cm³, such as a volume of at least about 5 cm³.

70. The delivery device of claim 68 or claim 69, wherein the capsule is spherical.

71. The delivery device of claim 68 or claim 69, wherein the capsule is oblong, egg shaped, oval and/or flattened.

72. The delivery device of any one of claims 68-71, wherein the capsule has a smallest diameter of at least about 5 mm, such as at least about 1 cm, such as at least about 2 cm.

73. The delivery device of any one of claims 67-71, wherein the capsule contains in its cavity a drug for treating symptoms of alzheimers, such as T3D-959, donepezil, galantamine, memantine, rivastigmine or any combinations thereof, preferably the drug is trapped in liposomes.

74. The delivery device of any one of claims 67-71, wherein the capsule is adapted for use in waste water treatment or in fuel-cells.

75. A delivery device suitable for delivering a chemical substance comprising one or more chemical compounds, the delivery device comprises a closed cavity comprising said chemical substance, the cavity is defined by an innermost wall surface, wherein at least a section of the inner wall surface constitutes an inner surface of a delivery membrane wherein the delivery membrane comprises an interpenetrating polymer network substrate comprising a host polymer and a guest polymer, where the guest polymer is interpenetrating the host polymer to form substantially continuous pathways within said host polymer.

76. A delivery device of claim 75 wherein the delivery device is according to any one of claims 1-74 wherein said chemical substance has been filled into said closed cavity.
